(19) United States
(12) Patent Application Publication
(10) Pub. No.: US 2025/0353892 A1
(43) Pub. Date: Nov. 20, 2025

(54) VIRUS-SPECIFIC RECOMBINANT T CELL RECEPTORS AND T CELLS COMPRISING THEM

(71) Applicants: SHEBA IMPACT LTD., Ramat Gan (IL); BAR-ILAN UNIVERSITY, Ramat Gan (IL); MINISTRY OF HEALTH, Jerusalem (IL)

(72) Inventors: Gal CAFRI, Nir David (IL); Nira VARDA-BLOOM, Hod Hasharon (IL); Elad JACOBY, Tel Aviv (IL); Amihai LIEBERMAN, Atlit (IL); Neta ZUCKERMAN, Tel Aviv (IL); Ayal HENDEL, Kiryat Ono (IL); Nimrod BEN HAIM, Holon (IL)

(21) Appl. No.: 18/871,595

(22) PCT Filed: Jun. 6, 2023
(86) PCT No.: PCT/IL2023/050581

§ 371 Date: Dec. 4, 2024

Related U.S. Application Data
(60) Provisional application No. 63/350,058, filed on Jun. 8, 2022.

(51) Int. Cl. C07K 14/725; A61K 40/11; A61K 40/32; A61K 40/46; C12N 5/0783

(52) U.S. Cl. CPC C07K 14/7051; A61K 40/11; A61K 40/32; A61K 40/46; C12N 5/0636; C12N 2510/00

(57) ABSTRACT

Provided herein are compositions comprising T cells expressing a recombinant virus-specific T cell receptor (rTCR-V) specific for a viral antigen restricted by a predetermined HLA type, rTCR-Vs, methods for preparing them, their use for treatment, and libraries containing such rTCR-V or T cells comprising them.

### FIELD OF THE INVENTION

The present is generally directed to recombinant T cell receptors for targeting cells infected with microorganisms, more specifically with viruses. In particular, the invention is directed to compositions comprising recombinant TCRs directed against viruses in a desired HLA background and uses thereof for treating viral infections in subjects.

### BACKGROUND OF THE INVENTION

Advanced therapies that expose patients to intensive antitumor chemotherapy, allogeneic hematopoietic stem cell transplantation (allo-HSCT), and solid organ transplantation (SOT) cause significant, life-threatening immune suppression. This immunocompromised state exposes patients to opportunistic, seasonal, and environmental infections, as well as viral reactivation that significantly increases morbidity and mortality rates and severely affects the outcome of alloHSCT and SOT. The ability to combat these threats mostly depends on post-transplant immune reconstitution which is a complex process that donor, recipient, and iatrogenic factors modulate. While innate immunity takes between 1 and 3 months to recover, adaptive immunity can take one year or more. In particular, the resulting B and T cell lymphopenia renders the transplant recipient vulnerable to opportunistic viral infections. These infections are the leading cause of transplant-related high morbidity and mortality rates. According to the European Society for Blood and Marrow Transplantation (EBMT), bacterial, fungal, and viral infection incidences may occur after auto-HSCT in 5-10% of patients, but with an increased rate of 20-50% following allo-HSCT, haplo-HSCT, and cord blood transplantation. The disease incidence increase in the pre-engraftment period is elongated according to the HLA matching status between donor and patient.

Conventional antiviral and antifungal pharmacotherapies have many limitations, including drug toxicity and the emergence of resistant infectious organisms. Antiviral agents such as ganciclovir, valganciclovir, cidofovir, and others have been shown to cause high rates of pancytopenia, neutropenia, and nephrotoxicity among bone marrow transplant patients. Antifungal agents such as amphotericin B, azoles, and echinocandins have severe adverse effects on renal and hepatic function. Moreover, the incidence of antiviral and antifungal resistance is rising, and chronic infection has been linked to T cell exhaustion and dysfunction.

Adoptive cell therapy explicitly directed against the pathogen target molecules is an emerging modality to effectively reduce or prevent the clinical manifestation of viral infections in immune-compromised patients. Donor-derived T cells targeting viral peptides were shown to be safe and effective. However, the personalized nature of this approach and the requirement for virus immune matched donors have emerged as barriers across the diversity of HLA alleles and the resulting number of potential genotypic combinations for under-represented ethnic and racial backgrounds patients. HLA haplotypes are geographically associated with very high diversity and therefore the risk of graft-versus-host disease (GvHD) due to allogeneic T-cells toxicity is a major safety concern. Furthermore, T cell products generally have a relatively low specificity against viral antigens.

Thus, there is a need in the art for more effective, specific, and safe T cell products to specifically target cells in a variety of individuals infected by a variety of viruses.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

According to some embodiments, there are provided herein compositions and methods to specifically target virally infected cells, and in particular, human cells infected with various types of viruses. In some embodiments, the compositions include T cells comprising a recombinant T cell receptor (TCR) which is specific to at least one viral antigen restricted by a predetermined HLA background.

In some embodiments, there is provided a composition comprising T cells expressing a recombinant virus-specific T cell receptor (rTCR-V) specific for a viral antigen restricted by a predetermined HLA type.

In some embodiments, at least 90% of the cells in the composition express the rTCR-V. In some embodiments, the T cells comprise CD8⁺ T cells. In some embodiments, the T cells comprise CD4⁺ T cells.

In some embodiments, the T cells do not express an endogenous TCR. In some embodiments, the T cells comprise a knocked-out (KO) TCR alpha constant (TRAC) locus.

In some embodiments, the T cells have an HLA type different from the predetermined HLA type.

In some embodiments, the rTCR-V comprises only sequences exogenous to the T cells. In some embodiments, the rTCR-V comprises a TCR sequence endogenous to the T cell in which a variable region sequence is replaced by a corresponding virus-specific TCR sequence exogenous to the T cell, the exogenous sequence comprising at least a virus-specific alpha chain CDR3 region sequence and/or a virus-specific beta chain CDR3 region sequence. In some embodiments, a sequence encoding the exogenous sequence is inserted into the TRAC locus on the T cells.

In some embodiments, the predetermined HLA type is selected from HLA-A2:01, HLA-A1:01, HLA-B7:02, HLA-A3:01, HLA-B8:01, HLA-B44:02, HLA-A24:02, HLA-B15:01, HLA-B51:01, HLA-A11:01, HLA-B35:01, HLA-B27:05, HLA-DPB1*: 04:01, HLA-DRB1*01:01, HLA-DRB1*03:01, HLA-DRB1*04:01, HLA-DRB1*07:01, HLA-DRB1*08:02, HLA-DRB1*11:01, HLA-DRB1*13:01, HLA-DRB1*15:01, HLA-DQB1*02:01, HLA-DQB1*03:01, HLA-DQB1*05:01, and HLA-DQB1*06:02. In some embodiments, the predetermined HLA type is selected from HLA A 02:01 and DPB1*: 04:01.

In some embodiments, the viral antigen is derived from a virus selected from adenovirus (ADV), cytomegalovirus (CMV), BK virus (BKV), John Cunningham virus (JC), Epstein-Barr virus (EBV), human herpes virus 6 (HHV6), and human immunodeficiency virus (HIV). In some embodiments, the virus is selected from ADV, CMV, or BKV.

In some embodiments, the virus is ADV. In some embodiments, the rTCR-V has alpha and beta chains CDR3 regions comprising the sequences defined by SEQ ID Nos. 1 and 2, respectively. In some embodiments, the rTCR-V has alpha and beta chains variable regions comprising the sequences set forth in SEQ ID Nos: 7 and 8, respectively.

In some embodiments, the virus is CMV. In some embodiments, the predetermined HLA type is HLA A 02:01 and DPB1*: 04:01. In some embodiments, the rTCR-V is specific to a CMV pp65 protein and the predetermined HLA type is HLA A 02:01 and DPB1*: 04:01. In some embodiments, the rTCR-V has alpha and beta chains CDR3 regions comprising the sequences defined by SEQ ID NOs. 3 and 4, respectively. In some embodiments, the rTCR-V has alpha and beta chains variable regions comprising the sequences defined by SEQ ID Nos: 9 and 10, respectively.

In some embodiments, rTCR-V is specific to a CMV pp65 protein peptide comprising a sequence as defined in SEQ ID NO: 22, and the predetermined HLA type is HLA A 02:01. In some embodiments, the rTCR-V has alpha and beta chains CDR3 regions comprising the sequences defined by SEQ ID NOs. 5 and 6, respectively. In some embodiments, the rTCR-V has alpha and beta chains variable regions comprising the sequences defined by SEQ ID Nos: 11 and 12, respectively.

In some embodiments, there is provided a recombinant virus-specific T cell receptor (rTCR-V) comprising six CDRs having sequences selected from: SEQ ID NOs 25, 26, 1, 27, 28, and 2; SEQ ID NOs 29, 30, 3, 31, 32, and 4; and SEQ ID NOs 33, 34, 5, 35, 36, and 6.

In some embodiments, there is provided recombinant virus-specific T cell receptor (rTCR-V) comprising alpha+beta variable region sequences selected from: SEQ ID NOs: 7+8; SEQ ID NOs: 9+10; and SEQ ID NOs: 11+12.

In some embodiments, there is provided a nucleic acid molecule comprising a nucleotide sequence encoding the recombinant virus-specific T cell receptor (rTCR-V) disclosed herein.

In some embodiments, the nucleic acid molecule comprises nucleotide sequences selected from: SEQ ID Nos: 13+14; SEQ ID NOs: 15+16; SEQ ID NOs: 17+18; SEQ ID No: 19; SEQ ID No: 20; and SEQ ID No: 21.

In some embodiments, there is provided a vector comprising the nucleic acid molecule disclosed herein.

In some embodiments, there is provided a host cell expressing the recombinant virus-specific T cell receptor (rTCR-V) disclosed herein, or comprising the nucleic acid molecule disclosed herein or the vector disclosed herein.

In some embodiments, the composition disclosed here or a composition comprising the host cell disclosed herein further comprise a pharmaceutically acceptable carrier.

In some embodiments, there is provided a composition as disclosed herein, for use in a method of treating or preventing viral infection in a subject, by administering to the subject a therapeutically effective amount of the composition.

In some embodiments, the treating or preventing is conducted by adoptive cell therapy (ACT). In some embodiments, the administration is following transplantation of an organ or cells from a transplantation donor, such as a hematopoietic stem cell transplantation (HSCT) or a solid organ transplantation (SOT) from a donor.

In some embodiments, the T cells are not derived from the subject or the transplantation donor.

In some embodiments, the subject is infected with a virus selected from ADV, CMV, BKV, JC, EBV, HHV6, and HIV.

In some embodiments, there is provided a method for preparing T cells expressing a recombinant virus-specific T cell receptor (rTCR-V) specific for a viral antigen restricted by a predetermined HLA type, the method comprising the steps of:
a) providing at least one virus-specific nucleotide sequence derived from a variable region of a T cell receptor (TCR) from a T cell specific to the viral antigen restricted by the predetermined HLA type;
b) preparing a nucleic acid molecule comprising the at least one virus-specific nucleotide sequence and encoding a partial or a complete a rTCR-V alpha and/or beta chain; and
c) introducing the nucleic acid molecule into host T cells, thereby obtaining T cells expressing a rTCR-V specific for a viral antigen restricted by a predetermined HLA type.

In some embodiments, the at least one virus-specific nucleotide sequence comprises a CDR3 region of the TCR. In some embodiments, the at least one virus-specific nucleotide sequence comprises a variable region of the TCR.

In some embodiments, providing the at least one virus-specific nucleotide sequence is conducted by the following steps:
1. providing precursor cells of the predetermined HLA type;
2. providing (i) at least one stimulating antigen derived from the at least one viral antigen, or (ii) antigen presenting cells (APCs) of the predetermined HLA type presenting at least one viral peptide derived from the viral antigen;
3. in vitro stimulating (IVS) the precursor cells by incubating with the APCs or with the at least one stimulating antigen of step 2 to obtain virus-specific stimulated T cells (VSTs);
4. isolating from the VSTs individual T cells specific for the viral antigen; and
5. sequencing at least part of a variable region from at least one TCR from the individual T cells specific to the viral antigen, thereby providing at least one virus-specific nucleotide sequence.

In some embodiments, providing antigen presenting cells (APCs) of the predetermined HLA type presenting at least one viral peptide derived from the viral antigen is conducted by the steps of: obtaining APCs from a sample of a donor having the predetermined HLA type; and incubating the APCs with the stimulating antigens derived from the viral antigen; thereby obtaining APCs presenting at least one viral peptide derived from the viral antigen.

In some embodiments, incubating is conducted in the presence of IL2.

In some embodiments, the APCs and the precursor cells are from the same source.

In some embodiments, isolating individual T cells specific for the viral antigen is conducted by the following steps: reactivating the stimulated T cells with stimulating antigens derived from the viral antigen or with APCs presenting viral peptides derived from the viral antigen; detecting and isolating T cells specific for the viral antigen by their expression of activation markers; and plating each isolated T cell individually; thereby obtaining individual T cells specific for the viral antigen.

In some embodiments, the activation markers are 4-1BB and/or OX40.

In some embodiments, the method further comprises a step of knocking-out the endogenous TCR of the host T cells.

In some embodiments, there are provided T cells expressing a recombinant virus-specific T cell receptor (rTCR-V) specific for a viral antigen restricted by a predetermined HLA type prepared by the method disclosed herein.

In some embodiments, there is provided a method of treating or preventing a viral disease in a subject, the method comprising administering to the subject a therapeutically effective amount of the composition disclosed herein or the T cells disclosed herein.

In some embodiments, there is provided a library comprising a plurality of T cells expressing a recombinant virus-specific T cell receptor (rTCR-V), each of the T cells as disclosed herein, wherein the library comprises at least two rTCR-Vs which are specific for viral antigens derived from the same virus, but the viral antigens are restricted by a different HLA type in each of the two rTCR-Vs.

In some embodiments, there is provided a library comprising a plurality of recombinant virus-specific T cell receptor (rTCR-Vs), each as disclosed herein, wherein the library comprises at least two rTCR-Vs which are specific for viral antigens derived from the same virus, but the viral antigens are restricted by a different HLA type in each of the two rTCR-Vs.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1 is schematic illustrations of the preparation of compositions which include virus-specific T cells (VSTs) having recombinant T cell receptors (TCRs), according to some embodiments.

FIGS. 2A-2D show the results of in vitro stimulation of T cells with pathogen-specific peptides. PBMCs were in vitro stimulated with APCs loaded with a CEF HLA class I control peptide pool (CEFX™) twice (IVS1, FIG. 2C, and IVS2, FIG. 2D). Cells were restimulated and tested for the surface expression of 4-1BB using flow cytometry. APCs without peptide (no peptide, FIG. 2A) and non-stimulated PBMCs (No IVS, FIG. 2B) served as negative controls.

FIGS. 3A-3E show In vitro stimulation of T cells with adenovirus (ADV) specific peptide libraries. PBMC were in vitro stimulated with APCs loaded with the antigens: hexon, penton, or hexon+penton-specific peptide pools. Unstimulated and CEF stimulated PBMC served as negative and positive controls, respectively. Cells were then restimulated with the above antigens and PMA/ION—non-specific stimulation cocktail (positive control), and tested for the surface expression of OX40 and 4-1BB (CD3⁺4-1BB⁺OX40⁺ cells—checkered bars, left bar each pair) using flow cytometry and for secretion of IFNγ by ELISA (IFN, hatched bars, right bar each pair). Each graph shows the specific T cell population that was enriched by a specific peptide library against: no antigen (FIG. 3A); CEF peptide pool (FIG. 3B), hexon (ADV) (FIG. 3C); penton (ADV) (FIG. 3D), and hexon+penton (ADV) (FIG. 3E).

FIGS. 4A-4D show testing or the IVS product by a co-culture with donor APCs loaded by the indicated antigen: FIG. 4A. IVS with no peptide. FIG. 4B; IVS with adenovirus peptides (Hexon library); FIG. 4C. IVS with BKV peptides VP1+LTA; FIG. 4D. IVS with CMV peptide (pp65); The T cells were stained for CD3 and for activation markers 4-1BB and OX40 and analyzed by FACS (black bars, left bar each pair), and the supernatant was analyzed for IFNγ level by ELISA (gray bars, right each pair). Adenovirus peptides: Hexon; BKV peptides: VP1+LTA; CMV peptides: pp65; triple: Hexon+VP1+LTA+pp65. PMA/ION—positive control.

FIGS. 5A-5L show identifying T cell clones specific for cytomegalovirus (CMV) and ADV for T cell receptor sequencing. PBMC were in vitro stimulated with APCs loaded with hexon (ADV) and pp65 (CMV) peptide pools. Unstimulated PBMC served as a negative control. Cells were then restimulated and sorted by FACS for activated CD4⁺ cells based on 4-1BB and OX40 expression. ADV-specific T cells: FIG. 5A: no stimulation; FIG. 5B: no peptide (neg control); FIG. 5C: hexon peptide library; FIG. 5D: PMA/ION positive control. CMV-specific T cells: FIG. 5E: no stimulation; FIG. 5F: no peptide (neg control); FIG. 5G: CMV peptides; FIG. 5H: PMA/ION positive control. FACS analysis of the T cell cultures is shown. FIG. 5I presents IFNγ expression of the restimulated ADV (left panel) and CMV (right panel) clones. FIGS. 5J-5L presents activation (4-1BB and OX40 expression as measured by FACS) of T cells comprising TCRs reconstructed based on sequences of reactive clones. FIG. 5J: no TCR; FIG. 5K: TCR specific to CMV-pp65 (see Table 3, clone 2), FIG. 5L: TCR specific to CMV-pp65 minimal epitope of SEQ ID NO: 22 (see Table 3, clone 3); left panels: CD4⁺ T cells, right panels CD8⁺ T cells.

FIG. 6 shows infection of donor-derived monocytes with different ADV subtypes. Donor PBMC were rested for two hours, and non-adherent cells were dispensed. ADV samples were added at different multiplicity of infection (MOI) levels. No. of ADV copies was measured at 24-48 hours post-infection using real-time PCR. Dark filled circles: control (no virus); light squares: ADV GFP MOI 1000; light triangles: ADV GFP MOI 3000; dark upside-down triangles: ADV5 MOI 0.001; dark diamonds: ADV3 MOI 0.5.

FIGS. 7A-7B show depletion of CD3 in T cells by CRISPR. T Cells were purified from PBMCs and stimulated for two days. On day 2 cells were electroporated with a CRISPR/Cas9 RNP complex targeting TRAC locus. Cells were stained on day 7 with anti-CD3 antibodies and % cells expressing CD3 was measured by flow cytometry. FIG. 7A shows a mock experiments, where 97.3% of the T cells express CD3, and FIG. 7B shows the results of the CD3 depletion, where only 1.05% of the T cells express CD3.

FIGS. 8A-8B show a mixed lymphocyte reaction (MLR) for tracking alloresponse rate. In FIG. 8A, PBMCs (responder) were stained with a fluorescent cell staining dye (carboxyfluorescein succinimidyl ester (CFSE)) and co-culture with irradiated PBMCs from a different subject (stimulator) in 3 different responder-stimulator (R:S) ratios (left to right: 2:1, 1:1, 1:2, middle panel). CFSE staining loss represents T cells in a proliferation stage. Positive control (upper panel)—responder cells with PHA. Negative control (lower panel)—responder only. FIG. 8B shows quantification of proliferation rates from FIG. 8A. Percentages of negative CFSE labeled cells, representing cells in proliferating stage, are presented. The three ratios are left to right: 2:1, 1:1, 1:2, and for each ratio, left to right: responder only, responder+stimulator, responder+PHA.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

The present invention generally provides pathogen-specific recombinant T cells and compositions including them for allogeneic use as antimicrobial therapies, such as antiviral, antibacterial or antifungal therapies. More specifically, the present invention is directed to T cells comprising a recombinant virus-specific TCR in a desired HLA background, suitable for administration to post-transplantation patients infected with a virus. The present invention is also directed to libraries comprising the recombinant T cells with various specificities restricted by various HLA backgrounds, so as to provide an ad hoc solution to a subject in need without a risk of graft versus host disease (GvHD).

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “a” and “an” refers to one or to more than one (i.e., to at least one) of the grammatical object of the article. By way of example, “an element” means one element or more than one element.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 90% and 110% of the given value.

For purposes of clarity, and in no way limiting the scope of the teachings, unless otherwise indicated, all numbers expressing quantities, percentages or proportions, and other numerical values recited herein, should be interpreted as being preceded in all instances by the term “about”, regardless of whether “about” is explicitly prepended to the numerical value. Accordingly, the numerical parameters recited in the present specification are approximations that may vary depending on the desired outcome. For example, each numerical parameter may be construed in light of the number of reported significant digits and by applying ordinary rounding techniques.

Accordingly, unless indicated to the contrary, the numerical parameters set forth in this specification are approximations that may vary by up to plus or minus 10% depending upon the desired properties to be obtained by the present invention.

It is further clarified that for any list of values that is preceded by a phrase such as “at least”, “about”, or “at least about”, each value in the list is interpreted to also be preceded by the same phrase preceding the first value.

In the description and claims of the application, the words “include” and “have”, and forms thereof, are not limited to members in a list with which the words may be associated. As used herein, the term comprising includes the term consisting of.

As used herein, according to some embodiments, the terms “substantially” and “about” may be interchangeable.

As referred to herein, the terms “nucleic acid molecule(s)” and “nucleotide molecule(s)” relate to polymers of deoxyribonucleotides (DNA), ribonucleotides (RNA), or modified forms thereof, in the form of a separate fragment or as a component of a larger construct, linear or branched, single stranded (ss), double stranded (ds), triple stranded (ts), or hybrids thereof. The term also encompasses RNA/DNA hybrids. The molecule may be, for example, a sense or antisense oligonucleotide or polynucleotide sequences of DNA or RNA. The DNA or RNA molecules may be, for example, but not limited to: complementary DNA (cDNA), genomic DNA, synthesized DNA, recombinant DNA, or a hybrid thereof or an RNA molecule such as, for example, mRNA, shRNA, siRNA, miRNA, and the like.

It is further noted that the phrase “a nucleic acid molecule comprising at least one nucleotide sequence encoding . . . ” is meant to also encompass a case where the nucleotide sequence may be divided between more than a single nucleic acid molecule. For example, since a T cell receptor (TCR) includes two chains (alpha and beta), a nucleotide sequence encoding a TCR may be divided into two nucleic acid molecules, each encoding one of the chains. However, for purposes of simplification, this situation is also intended to be covered by this phrase.

The terms “peptide” and “protein” are used herein to refer to polymers of amino acid residues. Generally, “peptide” relates to a short polymer of amino acid residues (as detailed below), while “protein” generally relates to a complete protein. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the peptide or the protein can contain modifications, such as but be not limited only to, glycosylation, phosphorylation or disulfide bond shape.

The term “sequence”, when used herein without indication of whether it is a nucleotide sequence or an amino acid sequence, may either be understood from the context, or, when appropriate, it may relate to both amino acid and to nucleic acid sequences. For example, CDR3 sequence may relate to either CDR3 amino acid sequence of the TCR polypeptide, or to CDR3 sequence of the TCR gene, or to both.

With regard to conservative substitution of amino acid sequences, one of skill will recognize that individual substitutions, deletions or additions to a nucleic acid, peptide, polypeptide, or protein sequence which alters, adds, or deletes a single amino acid or a small percentage of amino acids in the encoded sequence is a conservatively modified variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The term “construct”, as used herein refers to an artificially assembled or isolated nucleic acid molecule which may comprise one or more nucleic acid sequences, wherein the nucleic acid sequences may be coding sequences (that is, sequence which encode a protein or RNA product), regulatory sequences, non-coding sequences, or any combination thereof. The term construct includes without being limited to, for example, vectors and plasmids.

The term “recombinant” is used herein to describe molecules (such as, for example, nucleic acid molecules or polypeptide molecules) which have been synthetically constructed or genetically engineered by any method or are derived from or expressed from a molecule which has been synthetically constructed or genetically engineered, and to cells including these molecules. This term also encompasses molecules which have a sequence identical to a natural sequence.

As used herein, the terms “introducing”, such as, e.g., transfecting, refers to the transfer of molecules, such as, for example, nucleic acids, into a target cell. The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to heat shock, calcium phosphate transfection, PEI transfection, electroporation, lipofection, transfection reagent(s), viral-mediated transfer, injection, and the like, or combinations thereof. Transfection may be performed on any type of suitable cell, of any origin, such as, for example, human cells, animal cells, plant cells, and the like. The cells may be isolated cells, tissue cultured cells, cell lines, cells present within an organism body, and the like. In some embodiments, the cells are T cells.

As used herein, the term “in vitro” refers to an artificial environment and to processes or reactions that occur within an artificial environment. In vitro environments can consist of, but are not limited to, test tubes and cell culture. The term in vivo refers to the natural environment (e.g., an animal or a cell), and to processes or reactions that occur within a natural environment.

As used herein, the terms “subject”, “patient” or “individual” generally refer to a human, although the methods of the invention are not necessarily limited to humans and should be useful in other mammals.

As used herein, the term “donor” or “healthy donor” refers to a donor who provided a sample for preparing the recombinant T cell receptors and T cells of the invention. The donor may have provided the sample specifically for this purpose, or the donor may be a general donor, such as a blood donor, for which a sample was already available. In some embodiments, the donor is not infected with a respective virus. While blood donors must comply with certain health standards, it is not possible to know whether the donor or donors might have an undisclosed or unknown health condition.

The term “transplantation donor” refers specifically to a donor of transplanted organ or cells. In some embodiments, the donor is also the transplantation donor. In some embodiments, the donor is not the transplantation donor. In some embodiments, the donor is the subject who needs treatment.

The goal of the present invention is to provide to a subject infected with a virus, especially post-transplantation, T cells that can evoke an immune response against the virus, without a risk of graft versus host disease (GvHD). To this end, the present inventors developed recombinant virus-specific T cells having a recombinant T cell receptor (TCR) which specifically recognizes viral antigens in a desired human leukocyte antigen (HLA) background, and a process for obtaining such recombinant virus-specific T cells. Briefly, obtaining the recombinant virus-specific T cells of the invention involves preparing a recombinant TCR based on TCR sequences from T cells having a desired HLA background that are specific to viral antigens from the respective virus. Such recombinant, virus-specific TCRs (named herein rTCR-V)s may be prepared against a variety of viruses/viral antigens, and in a variety of HLA backgrounds, inserted into suitable vectors, and stored as a nucleic acid library until needed. When needed for treating a virus-infected subject, rTCR-Vs specific against the relevant virus and restricted by an HLA type matching that of the subject may be inserted into T cells (herein named rTCR-V T cells) in order to be administered to the subject. To avoid possible GvHD by the rTCR-V T cells, the endogenous TCR of the rTCR-V T cells may be knocked out. It is also conceivable that the rTCR-Vs are maintained in T cells, and the rTCR-V T cells are stored as a library until needed.

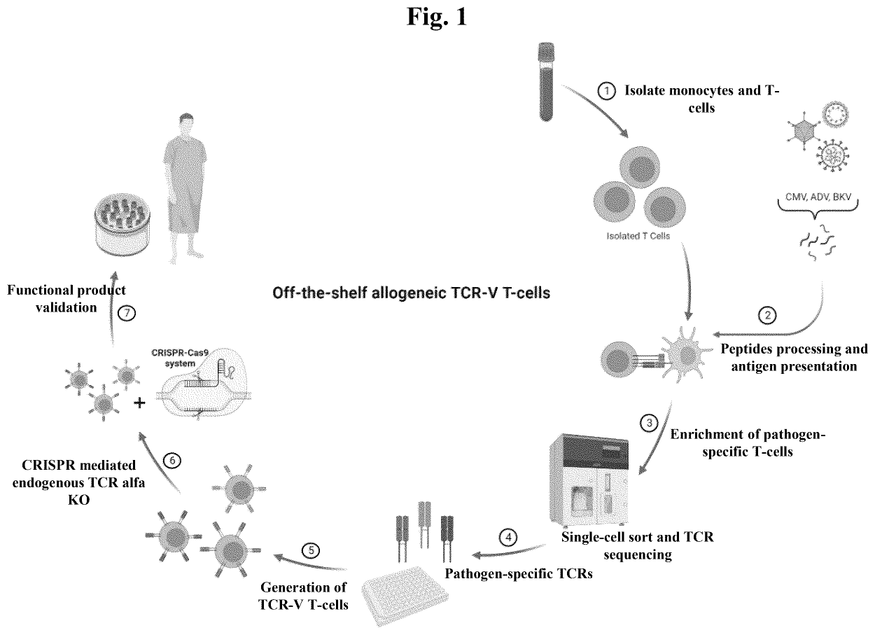

FIG. 1 is a schematic illustration generally describing a complete process of obtaining the rTCR-V T cells of the invention, according to certain embodiments. As shown in FIG. 1, the process starts with isolation of peripheral blood mononuclear cells (PBMCs) from blood of a donor in step (1), and optionally separating the T cells from the APCs. In the embodiment shown, the isolated T cells are challenged (in vitro stimulated, or IVS) in step (2) by antigen presenting cells (APCs) which were previously loaded with the desired antigens or peptides, such as antigens from adenovirus (ADV), cytomegalovirus (CMV), and/or BK-virus (BKV). It is noted, as also discussed below, that instead of incubating the donor PBMCs or T cells with loaded APCs presenting viral peptides, the PBMCs (which include T cells and APCs) may be directly incubated with the viral antigens, or with peptides derived therefrom.

Following the stimulation, virus-specific T cells (VSTs) become activated, as can be seen from the increased expression of activating markers such as 4-1BB and OX40, which may be used as shown in step (3) for enriching for and/or isolating the activated VSTs by flow cytometry. The isolated reactive VST clones specific to the antigen are sequenced and further tested as single cells for antigen recognition in step (4). TCR sequences from reactive clones are cloned into appropriate vectors and introduced in step (5) into T cells, in order to prepare recombinant virus-specific T cells (rTCR-V T cells). In some embodiments, a complete TCR sequence from the sequenced clones may be used. In some embodiments, a partial TCR sequence, such as a variable region sequence, or part thereof, may be used from the sequenced clone, and a full-length TCR gene including alpha and beta chains is prepared by adding TCR sequences from any suitable source. In step (6) the endogenous TCR gene of the rTCR-V T cells is knocked-out, in order to prevent graft versus host disease by the activity of the endogenous TCR. It is noted that the knocking out may take place before inserting the TCR into the cells. In some embodiments, only the TCR alpha chain is knocked out, which prevents assembly and expression of the endogenous TCR. In the demonstrated embodiment, the TCR alpha gene is knocked out by using a CRISPR-Cas9 system. The rTCR-V T cells product is validated, as shown in step (7), inter alia, by determining the antigen specificity and HLA restriction of the cells, and is ready for administering to a patient, or storage for future use.

In some embodiments, the present invention provides a composition comprising T cells expressing a recombinant microbe-specific T cell receptor specific for a microbial antigen restricted by a predetermined HLA type.

In some embodiments, the microbial antigen is selected from a viral antigen, a fungal antigen, or a bacterial antigen.

The description below relates mainly to target antigens which are viral antigens derived from viruses. However, the embodiments described below generally apply to other microorganisms, e.g., bacteria or fungi, mutatis mutandis.

As used herein, the term “viral antigen” refers to a antigen which is, or is derived from, a viral proteins. The viral antigen may be a complete protein or a partial protein, or a peptide derived from a viral protein.

In some embodiments, there is provided a composition comprising T cells expressing a recombinant virus-specific T cell receptor (rTCR-V) specific for a viral antigen restricted by a predetermined HLA type.

The terms “restricted by” and “presented by”, as used herein interchangeably with reference to a combination of a TCR (or T cells expressing the TCR), an antigen, and an HLA type, mean that the TCR recognizes peptides derived from the antigen and presented by HLA molecules of the indicated HLA type.

The T cells of the invention, expressing the rTCR-V, may also be referred to herein to as rTCR-V T cells.

As used herein, the term “T cell” refers to any type of T cell, including cells expressing CD3 (CD3⁺), CD8 (CD8⁺), CD4 (CD4⁺), and/or other relevant T cells markers. In some embodiments, T cells express at least CD3.

In some embodiments, the composition also comprises cells other than T cells, such as other immune cells. In some embodiments, the composition comprises mononuclear cells other than T cells, including but not limited to, monocytes, macrophages, and/or other antigen presenting cells, such as dendritic cells or macrophages. In some embodiments, the composition comprises only T cells. In some embodiments, the composition comprises only T cells specific for the viral antigen. In some embodiments, the composition comprises PBMCs.

According to some embodiments, at least 60%, at least 70%, at least 80%, at least 90%, at least 95%, or at least 97%, or at least 99% of the cells in the composition express the rTCR-V.

The terms “specific for” or “reactive to” used herein with respect to cells (e.g., T cells, CD3⁺ T cells, CD4⁺ T cells, CD8⁺ T cells, virus-specific cells) or TCRs, and an antigen (e.g., a viral antigen, a virus, a viral peptide), indicate the ability of the cells (or the TCRs) to be activated by the respective antigen, and therefore to be able to elicit an immune response against the antigen. Since the specificity of T cells is determined by the TCR they express, stating that a cell is specific for an antigen also means that the cell expresses a TCR that is specific for the antigen. When activated by the respective antigen (via the TCR), the reactive (specific) cells express activation markers such as 4-1BB and/or OX40, and/or secrete INFγ. Reactivity of the reactive cells may be defined by various methods. In some embodiments, reactivity of cells is defined by their expression of activating markers, such as, e.g., as may be determined by a fluorescence cell sorter (FACS) analysis, or by any other suitable method. In some embodiments, reactivity of cells is defined by their INFγ expression or secretion, such as by a specified dye or agent that specifically labels INFγ expressing cells.

T cells or TCRs specific for an antigen, or for a virus, are also referred to herein as “virus-specific”, as in virus-specific T cells, or a virus-specific TCR. It is further clarified that when T cells or TCRs are specific for a viral antigen, they are also said to be specific for the virus from which the viral antigen is derived.

According to some embodiments, at least 70%, at least 80%, at least 90%, at least 95%, at least 97%, or at least 99% of the cells in the composition are specific to the viral antigen.

In some embodiments, the composition includes at least about 10⁶, 10⁷, 10⁸, 10⁹, or 10¹⁰ T cells. For example, the composition may include from about 5×10⁵ to about 5×10⁶ T cells per ml, in a volume of from 50 to 200 ml. In some embodiments, the cells of the composition in various embodiments are at least 70% viable, and provided in a sterile medium, which may be a cryoprotectant medium (for example, 10% DMSO).

In some embodiments, T cells specific for the viral antigens further include T memory stem cells (Tscm). When administered to a subject, as described below with reference to methods of treatment, the composition therefore provides a durable response, including in vivo persistence of antigen-specific T cells for at least about 1 month, or at least about 3 months, or at least about 6 months, or at least about 12 months, or at least about 18 months, or at least about two years in some embodiments.

In some embodiments, the rTCR-V T cells comprise CD4⁺ T cells. In some embodiments, the rTCR-V T cells comprise CD8⁺ T cells. In some embodiments, the rTCR-V T cells comprise CD4⁺ T cells and CD8⁺ T cells.

In some embodiments, the rTCR-V T cells do not express an endogenous (innate) TCR. In some embodiments, the rTCR-V T cells express an endogenous TCR.

In some embodiments, the rTCR-V T cells comprise a knocked-out (KO) TCR alpha locus. In some embodiments, the rTCR-V T cells comprise a knocked-out (KO) TCR beta locus. In some embodiments, the rTCR-V T cells comprise a knocked-out (KO) TCR alpha and a knocked-out TCR beta locus. In some embodiments, the rTCR-V T cells comprise a knocked-out (KO) TCR alpha constant (TRAC) locus.

In some embodiments, the endogenous TCR gene of the rTCR-V T cells is knocked-out by a CRISPR-related system.

In some embodiments, the rTCR-V T cells have an HLA type different from the predetermined HLA type. In some embodiments, the rTCR-V T cells have an HLA type that is the same as the predetermined HLA type.

The rTCR-V is a virus-specific TCR encoded by at least one nucleotide sequence that may be referred to herein as a “rTCR-V encoding sequence”. The rTCR-V encoding sequence (and therefore the rTCR-V) may be completely exogenous to the T cell, or may be a chimeric sequence comprising both exogenous virus-specific TCR sequences and endogenous (innate) TCR sequences.

The term “endogenous”, as used herein, relates to the natural (or innate) TCR, or TCR encoding sequence, of the T cells comprising the rTCR-V.

The term “exogenous”, as used herein, relates to TCR sequences which are not derived from the endogenous TCR of the T cell. As used herein, the exogenous TCR sequences relate to amino acid sequences including, or nucleic acid sequences encoding the virus-specific sequences which render the rTCR-V with its viral antigen specificity.

In some embodiments, the exogenous rTCR-V encoding sequence(s) encode at least the CDR3 region(s) of the alpha and/or the beta chains of the rTCR-V. In some embodiments, the exogenous rTCR-V encoding sequence(s) encode at least the V-J region, including the CDR3 region, of the alpha chain of the rTCR-V, and/or the V-D-J region, including the CDR3 region, of the beta chain of the rTCR-V. In some embodiments, the exogenous rTCR-V encoding sequence(s) encode at least the alpha chain variable region and/or the beta chain variable region of the rTCR-V. In some embodiments, the exogenous rTCR-V encoding sequence(s) encode the complete rTCR-V.

Partial replacements of regions in the endogenous TCR of T cells with exogenous sequences may be carried out by various methods known in the art, including by CRISPR-cas9 and similar systems such as zinc finger nucleases (ZFNs), Transcription activator-like effector nuclease (TALEN), and megaTAL nucleases, or by homologous recombination methods, e.g., based on homology-directed repair (HDR).

Accordingly, in some embodiments, the rTCR-V comprises an endogenous TCR sequence in which a variable region sequence is replaced by a corresponding exogenous virus-specific TCR sequence, the exogenous virus-specific TCR sequence comprising at least a virus-specific alpha chain and/or a virus-specific beta chain CDR3 region sequence.

The term “corresponding” as used herein means that the exogenous virus-specific TCR sequence is from the same region (an analogous region) of a TCR as the variable region sequence of the endogenous TCR that is being replaced. For example, if the exogenous virus-specific TCR sequence encodes an alpha chain CDR3, then the endogenous variable region sequence also encodes an alpha chain CDR3.

In some embodiments, the exogenous virus-specific TCR sequence comprises a virus-specific alpha chain and/or virus-specific beta chain CDR3 region. In some embodiments, the exogenous virus-specific TCR sequence comprises a virus-specific alpha chain V-J and/or beta chain V-D-J region. In some embodiments, the exogenous virus-specific TCR sequence comprises a virus-specific alpha chain and/or a virus-specific beta chain variable region.

Accordingly, for example, in some embodiments, a CDR3 sequence of the endogenous alpha chain is replaced in the rTCR-V by an exogenous virus-specific alpha chain CDR3 sequence. In some embodiments, a CDR3 sequence of the endogenous beta chain is replaced in the rTCR-V by an exogenous virus-specific beta chain CDR3 sequence. Similarly, in some embodiments, a variable region sequence of the endogenous alpha and/or beta chain is replaced in the rTCR-V by an exogenous virus-specific alpha and/or beta chain variable region sequence, respectively.

In some embodiments, the rTCR-V does not comprise an endogenous sequence. In some embodiments, the rTCR-V comprises only exogenous TCR sequences.

In some embodiments, the exogenous virus-specific TCR sequence comprises at least one sequence selected from SEQ ID NOs: 1-12. In some embodiments, the exogenous virus-specific TCR sequence comprises at least one pair of sequences selected from SEQ ID NOs: 1+2; 3+4; 5+6; 7+8; 9+10; and 11+12. In some embodiments, the exogenous virus-specific TCR sequence comprises at least one group of CDR sequence selected from SEQ ID NOs: 25+26+1+27+28+2; 29+30+3+31+32+4; and 33+34+5+35+36+6.

It is noted that the exogenous sequence may relate to more than a single contiguous sequence.

In some embodiments, the exogenous virus-specific TCR sequence is encoded by a sequence selected from SEQ ID NOs: 13-18. In some embodiments, the exogenous virus-specific TCR sequence is encoded by a sequence selected from SEQ ID NOs: 13+14, 15+16, 17+18.

In some embodiments, the exogenous rTCR-V encoding sequence(s) is incorporated into the genomic DNA of the host T cell. In some embodiments, the exogenous rTCR-V encoding sequence(s) is incorporated into the endogenous TCR alpha or TCR beta genes. In some embodiments, the exogenous rTCR-V encoding sequence(s) is inserted into the TRAC locus on the T cells.

As used herein, the term “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells.

Adenovirus (ADV), Cytomegalovirus (CMV), and BK virus (BKV) are three viruses which are commonly reactivated in pediatric patients following bone marrow transplantations. While no effective treatment is available for ADV and BKV infection, an effective treatment is available for CMV infections. ADV causes a life-threatening infection to immunocompromised patients, and BKV infection causes a high risk for organ rejection and repetitive hospitalization.

As used herein, the terms “Adenovirus” and “ADV” are directed to members of the family Adenoviridae, which are medium-sized (90-100 nm), nonenveloped viruses with an icosahedral nucleocapsid containing a linear, non-segmented double stranded DNA genome (size of about 26-46 Kbp). Over 50 serotypes of ADV are known. ADV may cause respiratory, intestinal, and eye infections.

As used herein, the term “Cytomegalovirus” or “CMV” is directed to the genus of viruses in the order Herpesvirales, in the family Herpesviridae, in the subfamily Betaherpesvirinae. The 11 species in this genus include human betaherpesvirus 5 (HCMV, human cytomegalovirus, HHV-5), which is the species that infects humans. Diseases associated with HHV-5 include mononucleosis and pneumonia, and congenital CMV in infants can lead to deafness and ambulatory problems.

As used herein, the term “BKV” is directed to a BK virus of the polyomavirus family.

In some embodiments, the viral antigen is derived from a virus selected from ADV, CMV, BKV, John Cunningham virus (JC), Epstein-Barr virus (EBV), human herpesvirus 6 (HHV6), human immunodeficiency virus (HIV), and any combination thereof.

In some embodiments, the viral antigen is derived from ADV. In some embodiments, the viral antigen derived from is CMV. In some embodiments, the viral antigen is derived from BKV.

In some embodiments, the viral antigen is selected from the proteins hexon, penton, pp65, large T-antigen (LTA), and viral protein 1 (VP1). In some embodiments, the viral antigen is derived from the protein(s) hexon, penton, pp65, LTA, or VP1. In some embodiments, the viral antigen is, or is derived from, a hexon protein. In some embodiments, the viral antigen is, or is derived from, a penton protein. In some embodiments, the viral antigen is, or is derived from, a pp65 protein. In some embodiments, the viral antigen is, or is derived from, an LTA protein. In some embodiments, the viral antigens antigen is, or is from, a VP1 protein. In some embodiments, the hexon protein is an ADV hexon protein. In some embodiments, the penton protein is an ADV penton protein. In some embodiments, the pp65 protein is a CMV pp65 protein. In some embodiments, the LTA protein is a BKV LTA protein. In some embodiments, the VP1 protein is a BKV VP1 protein.

In some embodiments, all T cells in the composition express the same rTCR-V. In some embodiments, all T cells in the composition are specific for the same viral antigen. In some embodiments, all T cells in the composition are specific for the same virus.

In some embodiments, the composition comprises T cells expressing different rTCR-Vs. In some embodiments, the composition comprises T cells specific to different viral antigens. In some embodiments, the composition comprises T cells specific to different viral antigens derived from the same virus. In some embodiments, the composition comprises T cells specific to different viral antigens derived from the different viruses.

In some embodiments, the composition comprises T cells specific for viral antigens derived from more than one viruses. In some embodiments, the more than one viruses are selected from ADV, CMV, BKV, JC, EBV, HHV6, and HIV, and combinations thereof.

In some embodiments, the more than one viruses comprise ADV and/or CMV. In some embodiments, the more than one viruses comprise ADV and/or BKV. In some embodiments, the more than one viruses comprise CMV and/or BKV.

In some embodiments, the more than one viruses comprise ADV and CMV. In some embodiments, the more than one viruses comprise ADV and BKV. In some embodiments, the more than one viruses comprise CMV and BKV.

In some embodiments, the more than one viruses are ADV and CMV. In some embodiments, the more than one viruses are ADV and BKV. In some embodiments, the more than one viruses are CMV and BKV.

In some embodiments, the more than one viruses comprise ADV, CMV, and/or BKV. In some embodiments, the more than one viruses comprise ADV, CMV, and BKV. In some embodiments, the more than one viruses are ADV, CMV, and BKV.

In some embodiments, the virus is ADV, and the rTCR-V has alpha and beta chains CDR3 regions comprising sequences defined by SEQ ID NOs. 1 and 2, respectively.

In some embodiments, the viral antigen is ADV hexon protein, and the rTCR-V has alpha and beta chains CDR3 regions comprising sequences defined by SEQ ID NOs. 1 and 2, respectively.

In some embodiments, the virus is ADV, and the rTCR-V has alpha and beta chains variable regions comprising sequences defined by SEQ ID NOs. 7 and 8, respectively.

In some embodiments, viral antigen is ADV hexon protein, and the rTCR-V has alpha and beta chains variable regions comprising sequences defined by SEQ ID NOs. 7 and 8, respectively.

In some embodiments, the virus is ADV, and the rTCR-V has alpha and beta chains variable regions comprising sequences encoded by SEQ ID NOs. 13 and 14, respectively.

In some embodiments, viral antigen is ADV hexon protein, and the rTCR-V has alpha and beta chains variable regions comprising sequences encoded by SEQ ID NOs. 13 and 14, respectively.

In some embodiments, the viral antigen is ADV hexon protein, and the rTCR-V comprises a sequences encoded by SEQ ID No. 19.

In some embodiments, the virus is ADV or the viral antigen is ADV hexon protein, and the rTCR-V has six CDRs comprising the sequences defined by SEQ ID NOs. 25, 26, 1, 27, 28, and 2.

In some embodiments, the virus is CMV, and the rTCR-V has alpha and beta chains CDR3 regions comprising sequences defined by SEQ ID NOs. 3 and 4, respectively.

In some embodiments, the viral antigen is CMV pp65, and the rTCR-V has alpha and beta chains CDR3 regions comprising sequences defined by SEQ ID NOs. 3 and 4, respectively.

In some embodiments, the virus is CMV, and the rTCR-V has alpha and beta chains variable regions comprising sequences defined by SEQ ID NOs. 9 and 10, respectively.

In some embodiments, the viral antigen is CMV pp65, and the rTCR-V has alpha and beta chains variable regions comprising sequences defined by SEQ ID NOs. 9 and 10, respectively.

In some embodiments, the virus is CMV, and the rTCR-V has alpha and beta chains variable regions comprising sequences encoded by SEQ ID NOs. 15 and 16, respectively.

In some embodiments, the viral antigen is CMV pp65, and the rTCR-V has alpha and beta chains variable regions comprising sequences encoded by SEQ ID NOs. 15 and 16, respectively.

In some embodiments, the viral antigen is CMV pp65, and the rTCR-V comprises a sequences encoded by SEQ ID No. 20.

In some embodiments, the virus is CMV or the viral antigen is CMV pp65 protein, and the rTCR-V has six CDRs comprising the sequences defined by SEQ ID NOs. 29, 30, 3, 31, 32, and 4.

In some embodiments, the viral antigen is a CMV pp65 minimal epitope as defined in SEQ ID NO: 22, and the rTCR-V has alpha and beta chains CDR3 regions comprising sequences defined by SEQ ID NOs. 5 and 6, respectively.

In some embodiments, the viral antigen is a CMV pp65 minimal epitope as defined in SEQ ID NO: 22, and the rTCR-V has alpha and beta chains variable regions comprising sequences defined by SEQ ID NOs. 11 and 12, respectively.

In some embodiments, the viral antigen is a CMV pp65 minimal epitope as defined in SEQ ID NO: 22, and the rTCR-V has alpha and beta chains variable regions comprising sequences encoded by SEQ ID NOs. 17 and 18, respectively.

In some embodiments, the viral antigen is a CMV pp65 minimal epitope as defined in SEQ ID NO: 22, and the rTCR-V comprises a sequences encoded by SEQ ID No. 21.

In some embodiments, the virus is CMV or the viral antigen is CMV pp65 protein minimal epitope as defined in SEQ ID NO: 22, and the rTCR-V has six CDRs comprising the sequences defined by SEQ ID NOs. 33, 34, 5, 35, 36, and 6.

In some embodiments, the predetermined HLA type is selected from the HLA class I molecules: HLA-A2:01, HLA-A1:01, HLA-B7:02, HLA-A3:01, HLA-B8:01, HLA-B44:02, HLA-A24:02, HLA-B15:01, HLA-B51:01, HLA-A11:01, HLA-B35:01, and HLA-B27:05.

In some embodiments, the predetermined HLA type is selected from the HLA class II molecules: DPB1*: 04:01, HLA-DRB1*01:01, HLA-DRB1*03:01, HLA-DRB1*04:01, HLA-DRB1*07:01, HLA-DRB1*08:02, HLA-DRB1*11:01, HLA-DRB1*13:01, HLA-DRB1*15:01, HLA-DQB1*02:01, HLA-DQB1*03:01, HLA-DQB1*05:01, and HLA-DQB1*06:02.

In some embodiments, the predetermined HLA type is selected from the HLA molecules: HLA-A2:01, HLA-A1:01, HLA-B7:02, HLA-A3:01, HLA-B8:01, HLA-B44:02, HLA-A24:02, HLA-B15:01, HLA-B51:01, HLA-A11:01, HLA-B35:01, HLA-B27:05, DPB1*: 04:01, HLA-DRB1*01:01, HLA-DRB1*03:01, HLA-DRB1*04:01, HLA-DRB1*07:01, HLA-DRB1*08:02, HLA-DRB1*11:01, HLA-DRB1*13:01, HLA-DRB1*15:01, HLA-DQB1*02:01, HLA-DQB1*03:01, HLA-DQB1*05:01, and HLA-DQB1*06:02.

In some embodiments, the predetermined HLA type is selected from HLA A 02:01, DPB1*: 04:01.

In some embodiments, there is provided a composition comprising T cells expressing a recombinant ADV-specific rTCR-V comprising a TCR alpha chain CDR3 region comprising a sequence as set forth in SEQ ID NO: 1 and a TCR beta chain CDR3 region comprising a sequence as set forth in SEQ ID NO:2.

In some embodiments, there is provided a composition comprising T cells expressing a recombinant ADV-specific rTCR-V comprising a TCR alpha chain variable region comprising a sequence as set forth in SEQ ID NO: 7 and a TCR beta chain variable region comprising a sequence as set forth in SEQ ID NO:8.

In some embodiments, there is provided a composition comprising T cells expressing a recombinant CMV-specific rTCR-V comprising a TCR alpha chain CDR3 region comprising a sequence as set forth in SEQ ID NO: 3 and a TCR beta chain CDR3 region comprising a sequence as set forth in SEQ ID NO:4.

In some embodiments, there is provided a composition comprising T cells expressing a recombinant CMV-specific rTCR-V comprising a TCR alpha chain variable region comprising a sequence as set forth in SEQ ID NO: 9 and a TCR beta chain variable region comprising a sequence as set forth in SEQ ID NO:10.

In some embodiments, there is provided a composition comprising T cells expressing a recombinant CMV-specific rTCR-V comprising a TCR alpha chain CDR3 region comprising a sequence as set forth in SEQ ID NO: 5 and a TCR beta chain CDR3 region comprising a sequence as set forth in SEQ ID NO:6.

In some embodiments, there is provided a composition comprising T cells expressing a recombinant CMV-specific rTCR-V comprising a TCR alpha chain variable region comprising a sequence as set forth in SEQ ID NO: 11 and a TCR beta chain variable region comprising a sequence as set forth in SEQ ID NO:12.

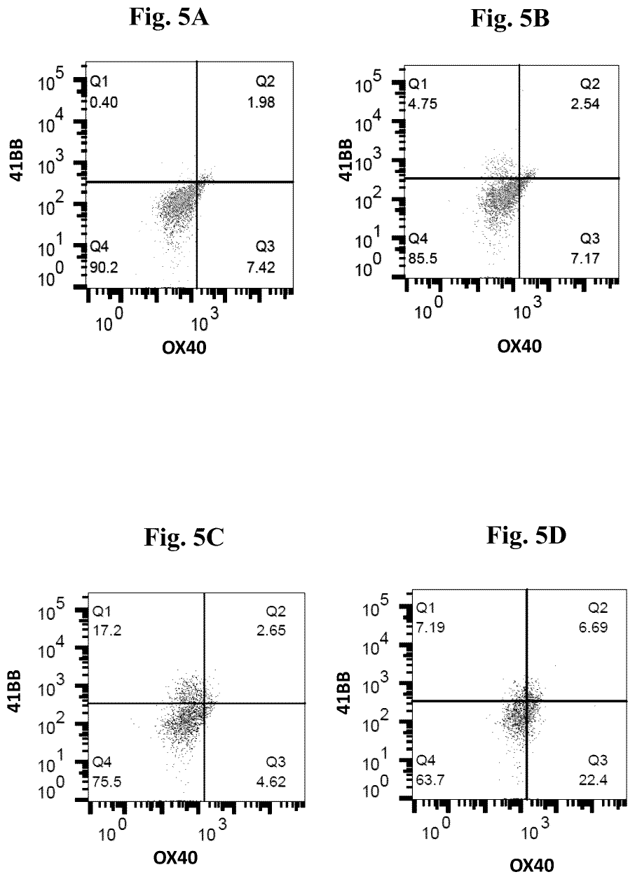

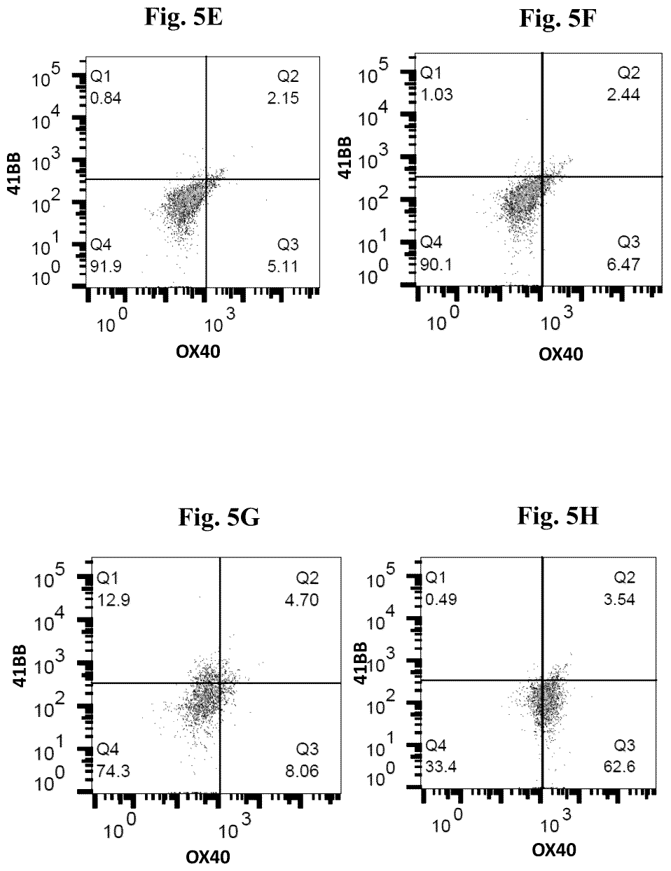

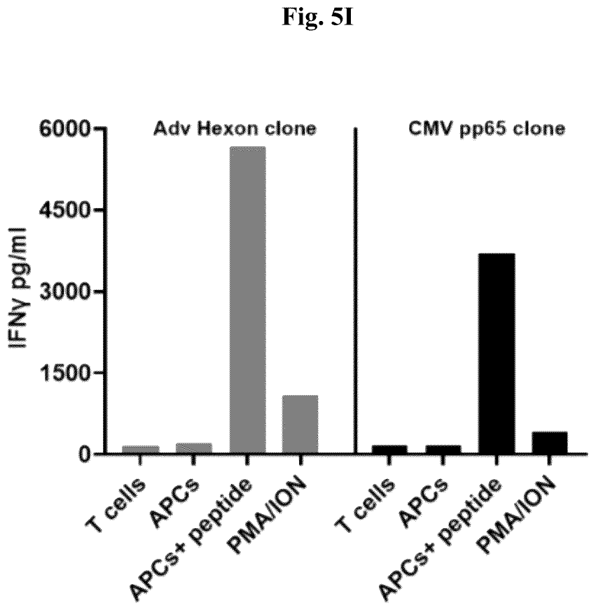

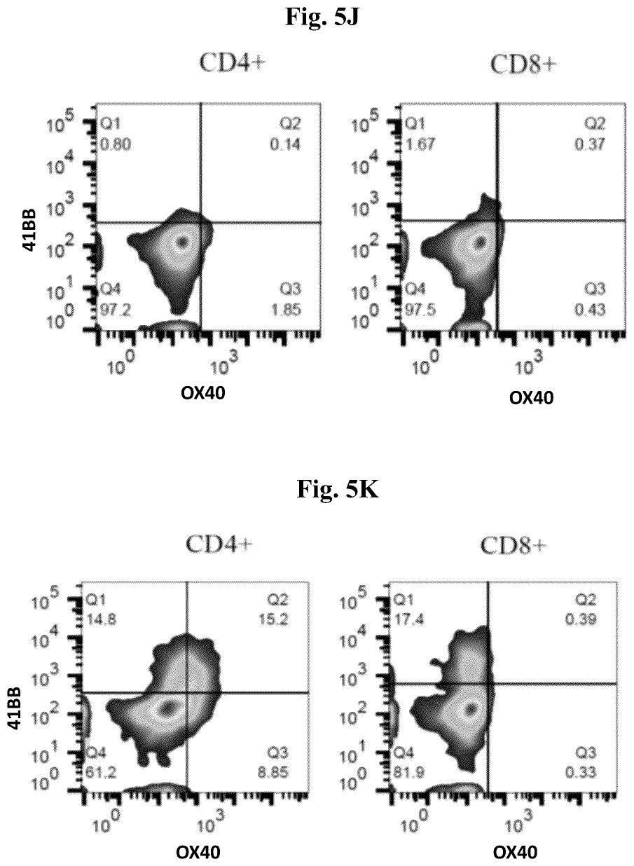

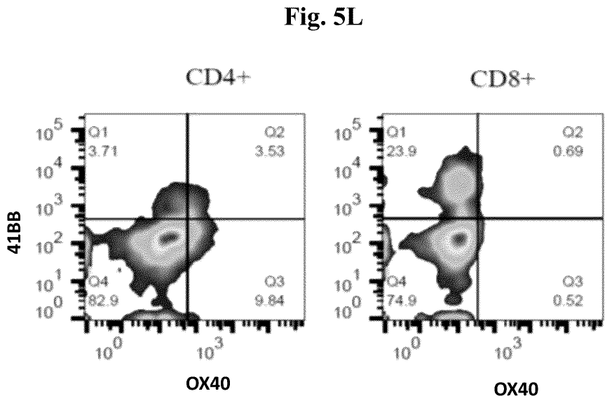

As detailed in Example 4, the methods described herein below led to generation of virus-specific T cells (VSTs), which were specific to antigens derived from ADV and CMV, including VSTs specific to ADV hexon protein, CMV pp65 protein, and to a specific epitope of the CMV pp65 protein, having a sequence set forth in SEQ ID NO: 22. FIGS. 5A-5I demonstrate the specificity and reactivity of selected clones, which were subsequently isolated and sequenced, to yield virus-specific sequences (see Tables 2, 3) which were then used to generate full length rTCR-Vs, as described in more detail below, and introduce it by retroviral transduction into T cells (rTCR-V T cells). The specificity and reactivity of selected rTCR-V T cells is shown in FIGS. 5J-5L.

TCRs are heterodimeric polypeptides expressed on the surface of T cells and determining their antigen specificity. Most TCRs comprise an alpha chain (TRA) and a beta chain (TRB), and a small percentage comprises gamma and delta chains. The alpha and beta chains include a constant region and a variable region. The variable regions of the alpha and beta chains further comprises 2-3 regions (V, D, and J for the beta chain, and V and J for the alpha chain). Each of the alpha and the beta chains comprises 3 complementarity determining regions (CDRs), of which, the CDR3 is the main CDR responsible for specific antigen recognition.

Antigens are presented to T cells by antigen presenting cells (as discussed further herein) which display peptides derived from the antigen on an HLA type I or type II molecule. Accordingly, a TCR specific to an antigen (e.g., the virus-specific TCR) may also be referred to as specific to a peptide derived from the antigen, presented by, or restricted by a specific HLA type.

In some embodiments, there is provided a recombinant virus-specific TCR (rTCR-V) specific for a viral antigen, such as viral antigens described herein, restricted by a predetermined HLA type.

As defined herein, the viral antigen may be a complete protein or a partial protein, or a peptide derived from a viral protein.

In some embodiments, the viral antigen is derived from a virus selected from ADV, CMV, BKV, JC, EBV, HHPV, HIV, and combinations thereof. In some embodiments, the viral antigen is derived from ADV, CMA, or BKV.

In some embodiments, the viral antigen is selected from an ADV hexon protein and a CMV pp65 protein. In some embodiments, the viral antigen is ADV hexon protein. In some embodiments, the viral antigen is CMV pp65 protein. In some embodiments, the viral antigen is an ADV hexon protein-derived peptide or epitope. In some embodiments, the viral antigen is a CMV pp65 protein-derived peptide or epitope. In some embodiments, the viral antigen is a CMV pp65 peptide having a sequence as set forth in SEQ ID NO: 22.

In some embodiments, predetermined HLA type selected from HLA A2 and DPB1. In some embodiments, predetermined HLA type selected from HLA A 02:01 and DPB1*: 04:01.

In some embodiments, the viral antigen is a CMV viral antigen presented by an HLA type II molecule DPB1*: 04:01, or an HLA type I molecule HLA A 02:01. In some embodiments, the viral antigen is a CMV viral antigen having a sequence as set forth in SEQ ID NO: 22, presented by an HLA class I molecule HLA A 02:01.

In some embodiments, the rTCR-V is specific to an ADV antigen or a CMV antigen restricted by an HLA class II HLA DPB1*: 04:01, or HLA class I HLA A 02:01.

In some embodiments, the rTCR-V is specific to a CMV antigen restricted by an HLA class II DPB1*: 04:01, or HLA class I HLA A 02:01. In some embodiments, the rTCR-V is specific to a CMV antigen having a sequence as set forth in SEQ ID NO: 22, restricted by HLA class I molecule HLA A 02:01.

In some embodiments, the rTCR-V comprises a TCR alpha chain variable region comprising a TRAV17*01 V region, a TRAJ57*01 J region, and a CDR3 having a sequence as set forth in SEQ ID NO: 1.

In some embodiments, the rTCR-V comprises a TCR beta chain variable region comprising a TRBV3-1*01V region, a TRBD2*01 D region, a TRBJ1-1*01 J region, and a CDR3 having a sequence as set forth in SEQ ID NO: 2.

In some embodiments, the rTCR-V comprises and a TCR alpha chain variable region comprising a TRAV17*01 V region, a TRAJ57*01 J region, and a CDR3 having a sequence as set forth in SEQ ID NO: 1; and a TCR beta chain variable region comprising a TRBV3-1*01V region, a TRBD2*01 D region, a TRBJ1-1*01 J region, and a CDR3 having a sequence as set forth in SEQ ID NO: 2.

In some embodiments, rTCR-V has a TCR alpha chain variable region according to SEQ ID NO: 7. In some embodiments, rTCR-V has a TCR beta chain variable region according to SEQ ID NO: 8. In some embodiments, rTCR-V has a TCR alpha chain variable region according to SEQ ID NO: 7, and a TCR beta chain variable region according to SEQ ID NO: 8.

In some embodiments, rTCR-V comprises six CDRs according to SEQ ID Nos: 25, 26, 1, 27, 28, and 2.

In some embodiments, rTCR-V has a TCR alpha chain variable region encoded by SEQ ID NO: 13. In some embodiments, rTCR-V has a TCR beta chain variable region encoded by SEQ ID NO: 14. In some embodiments, the rTCR-V has a TCR alpha chain variable region encoded by SEQ ID NO: 13, and a TCR beta chain variable region encoded by SEQ ID NO: 14.

In some embodiments, rTCR-V comprises a sequence encoded by SEQ ID NO: 19.

In some embodiments, the rTCR-V is specific to an ADV viral antigen. In some embodiments, the rTCR-V is specific to an ADV hexon protein.

In some embodiments, the rTCR-V comprises a TCR alpha chain variable region comprising a TRAV8-3*01 V region, a TRAJ15*01 J region, and a CDR3 having a sequence as set forth in SEQ ID NO: 3.

In some embodiments, the rTCR-V comprises a TCR beta chain variable region comprising a TRBV4-3*01 V region, a TRBD2*02 D region, a TRBJ1-1*01 J region, and a CDR3 having a sequence as set forth in SEQ ID NO: 4.

In some embodiments, the rTCR-V comprises a TCR alpha chain variable region comprising a TRAV8-3*01 V region, a TRAJ15*01 J region, and a CDR3 having a sequence as set forth in SEQ ID NO: 3; and a TCR beta chain variable region comprising a TRBV4-3*01 V region, a TRBD2*02 D region, a TRBJ1-1*01 J region, and a CDR3 having a sequence as set forth in SEQ ID NO: 4.

In some embodiments, rTCR-V has a TCR alpha chain variable region according to SEQ ID NO: 9. In some embodiments, rTCR-V has a TCR beta chain variable region according to SEQ ID NO: 10. In some embodiments, the rTCR-V has a TCR alpha chain variable region according to SEQ ID NO: 9, and a TCR beta chain variable region according to SEQ ID NO: 10.

In some embodiments, rTCR-V comprises six CDRs according to SEQ ID Nos: 29, 30, 3, 31, 32, and 4.

In some embodiments, rTCR-V has a TCR alpha chain variable region encoded by SEQ ID NO: 15. In some embodiments, rTCR-V has a TCR beta chain variable region encoded by SEQ ID NO: 16. In some embodiments, the rTCR-V has a TCR alpha chain variable region encoded by SEQ ID NO: 15, and a TCR beta chain variable region encoded by SEQ ID NO: 16.

In some embodiments, rTCR-V comprises a sequence encoded by SEQ ID NO: 20.

In some embodiments, the rTCR-V is specific to a CMV viral antigen. In some embodiments, the rTCR-V is specific to a CMV pp65 protein. In some embodiments, the rTCR-V is specific to a CMV viral antigen restricted by HLA class II molecule DPB1*: 04:01. In some embodiments, the rTCR-V is specific to a CMV pp65 protein restricted by HLA class II molecule DPB1*: 04:01.

In some embodiments, the rTCR-V polypeptide comprises a TCR alpha chain variable region comprising a TRAV24*01 V region, a TRAJ30*01 J region, and a CDR3 having a sequence as set forth in SEQ ID NO: 5.

In some embodiments, the rTCR-V polypeptide comprises a TCR beta chain variable region comprising a TRBV28*01 V region, a TRBD1*01 D region, a TRBJ2-7*02 J region, and a CDR3 having a sequence as set forth in SEQ ID NO: 6.

In some embodiments, the rTCR-V polypeptide comprises; and a TCR alpha chain variable region comprising a TRAV24*01 V region, a TRAJ30*01 J region, and a CDR3 having a sequence as set forth in SEQ ID NO: 5; and a TCR beta chain variable region comprising a TRBV28*01 V region, a TRBD1*01 D region, a TRBJ2-7*02 J region, and a CDR3 having a sequence as set forth in SEQ ID NO: 6.

In some embodiments, rTCR-V comprises six CDRs according to SEQ ID Nos: 33, 34, 5, 35, 36, and 6.

In some embodiments, rTCR-V has a TCR alpha chain variable region according to SEQ ID NO: 11. In some embodiments, rTCR-V has a TCR beta chain variable region according to SEQ ID NO: 12. In some embodiments, rTCR-V has a TCR alpha chain variable region according to SEQ ID NO: 11, and a TCR beta chain variable region according to SEQ ID NO: 12.

In some embodiments, rTCR-V has a TCR alpha chain variable region encoded by SEQ ID NO: 17. In some embodiments, rTCR-V has a TCR beta chain variable region encoded by SEQ ID NO: 18. In some embodiments, the rTCR-V has a TCR alpha chain variable region encoded by SEQ ID NO: 17, and a TCR beta chain variable region encoded by SEQ ID NO: 18.

In some embodiments, rTCR-V comprises a sequence encoded by SEQ ID NO: 21.

In some embodiments, rTCR-V is specific to a CMV viral antigen. In some embodiments, the rTCR-V is specific to a CMV pp65 protein. In some embodiments, the rTCR-V is specific to a CMV pp65 peptide having a sequence as set forth in SEQ ID NO: 22. In some embodiments, the rTCR-V is specific to a CMV viral antigen restricted by HLA class I molecule HLA A 02:01. In some embodiments, the rTCR-V is specific to a CMV pp65 protein restricted by HLA class I molecule HLA A 02:01. In some embodiments, the rTCR-V is specific to a CMV pp65 peptide having a sequence as set forth in SEQ ID NO: 22 restricted by HLA class I molecule HLA A 02:01.

In some embodiments, the rTCR-V comprises alpha and beta variable regions sequences selected from the pairs: SEQ ID NOs: 7 (alpha)+SEQ ID NOs: 8 (beta); SEQ ID NOs: 9 (alpha)+SEQ ID NOs: 10 (beta); and SEQ ID NOs: 11 (alpha)+SEQ ID NOs: 12 (beta).

In some embodiments, the rTCR-V comprises six CDRs having sequences selected from SEQ ID NOs: 25+26+1+27+28+2; 29+30+3+31+32+4; and 33+34+5+35+36+6.

It should be clarified that the “+” sign only indicates that all of the indicated CDRs are included in the rTCR-V, but not that they are necessarily part of a contiguous sequence or even in the same molecule (since a TCR, e.g., a rTCR-V usually includes two polypeptide chains encoded by separate genes). Usually there are sequences separating the different CDRs, and they may be included or encoded on separate molecules, which together form the rTCR-V or the sequences encoding it. The same is intended for other instances herein when a list of sequences (e.g., a list of CDRs or alpha and beta chains) is included in a sequence or a molecule (e.g., a rTCR-V)—the individual sequences listed as separate SEQ ID Nos. are not necessarily contiguous and are usually interrupted by other sequences. This applies for any type of sequences, such as amino acid or nucleic acid sequences.

In some embodiments, the present invention comprises a nucleic acid molecule including a nucleotide sequence encoding at least part of the rTCR-V of the invention. In some embodiments, the alpha chain and the beta chain are encoded on the same nucleic acid molecule. In some embodiments, the alpha chain and the beta chain are encoded on separate nucleic acid molecules.

In some embodiments, the rTCR-V includes human sequences. In some embodiments, the rTCR-V includes mouse sequences. In some embodiments, the rTCR-V is a chimeric TCR, including both human and mouse sequences. For example, in some embodiments, the rTCR-V comprises human CDRs with the rest of the sequences being derived from mouse, or human CDR3 with the rest of the sequences being derived from mouse. In some embodiments, the rTCR-V includes framework regions from mouse. In some embodiments, the rTCR-V includes framework regions from human. In some embodiments, the rTCR-V includes constant regions from mouse.

In some embodiments, the rTCR-V includes constant regions from human. Advantages of the mouse TCR sequences may include, e.g., reducing the amount of mispairing between the exogenous TCR chains of the invention and any endogenous TCR chains (for example, if not knocked-out).

In some embodiments, the nucleic acid molecule comprises rTCR-V sequences encoding the six CDRs defined by sequences selected from SEQ ID NOs: 25+26+1+27+28+2; 29+30+3+31+32+4; and 33+34+5+35+36+6.

In some embodiments, the nucleic acid molecule comprises rTCR-V sequences encoding sequences selected from the alpha+beta chain variable region sequence pairs: SEQ ID Nos: 7+8; SEQ ID NOs: 9+10; and SEQ ID NOs: 11+12.

In some embodiments, the nucleic acid molecule comprises rTCR-V sequences selected from the alpha+beta chain variable region sequence pairs: SEQ ID Nos: 13+14; SEQ ID NOs: 15+16; and SEQ ID NOs: 17+18.

In some embodiments, the nucleic acid molecule comprises complete rTCR-V sequences selected from SEQ ID No: 19; SEQ ID No: 20; and SEQ ID No: 21.

It is appreciated that although the CDR sequences are provided as amino acid sequences, it is a trivial task for a person skilled in the art to find nucleic acids encoding these sequences. Furthermore, as an example, since the nucleotide sequences of the variable chains encoding these CDRs are available in the present application, if needed, the nucleotide sequence can easily be found within these sequences.

In some embodiments, the nucleic acid molecule comprises rTCR-V sequences that are comprised in SEQ ID NO: 13 and encode SEQ ID Nos: 25, 26, and 1; and rTCR-V sequences that are comprised in SEQ ID NO: 14 and encode SEQ ID Nos: 27, 28, and 2.

In some embodiments, the nucleic acid molecule comprises rTCR-V sequences that are comprised in SEQ ID NO: 15 and encode SEQ ID Nos: 29, 30, and 3; and rTCR-V sequences that are comprised in SEQ ID NO: 16 and encode SEQ ID Nos: 31, 32, and 4.

In some embodiments, the nucleic acid molecule comprises rTCR-V sequences that are comprised in SEQ ID NO: 17 and encode SEQ ID Nos: 33, 34, and 5; and rTCR-V sequences that are comprised in SEQ ID NO: 18 and encode SEQ ID Nos: 35, 36, and 6.

In some embodiments, the present invention provides a vector comprising the nucleic acid molecule discloses herein.

As used herein the term “vector” refers to constructs engineered to deliver into, or encode or express in, a target cell, nucleic acid molecules, such as DNA, RNA, miRNA, shRNA, siRNA, and antisense oligonucleotides. Vectors may include, e.g., viral and non-viral vectors, γ-retroviral or lentiviral vectors. The term “Expression vector” refers to vectors that have the ability to incorporate and express heterologous nucleic acid fragments (such as DNA) in a cell. In other words, an expression vector comprises nucleic acid sequences/fragments (such as DNA, mRNA, tRNA, rRNA), capable of being transcribed or expressed in a target cell. Many viral, prokaryotic and eukaryotic expression vectors are known and/or commercially available. Selection of appropriate expression vectors is within the knowledge of those having skill in the art. Vectors may include functional elements required for the desired function of the nucleic acid in the cells, including, for example, a promoter suitable for expression in the target cell, targeting elements for incorporating the nucleic acid in a specific location in the target cell DNA, and replication sequences for replicating the vector.

In some embodiments, the vector is suitable for delivery of the nucleic acids of the invention into T cells, and optionally for expressing the TCR in the T cells. n some embodiments, the vector may be a vector suitable for inserting the TCR into the endogenous DNA of the T cell. In some embodiments, the vector is suitable for knocking-out the endogenous TCR and knocking-in the nucleic acid of the invention. In some embodiments, the vector may be suitable for facilitating integration of the nucleic acid of the invention into the TCR alpha constant (TRAC) locus. Vectors suitable for any of the above purposes are well-known in the art. For example, in some embodiments, the vector may be plasmid-based (e.g. pUC-based, pBluescript-based, etc.) a viral, or a retroviral vector, such as MSGV1.

In some embodiments, the vector is not directed for incorporation of the nucleotide sequence encoding at least part of the rTCR-V into a specific genomic region. In some embodiment, the vector comprises a promotor for driving expression of the rTCR-V.

In some embodiments, the vector is directed for incorporation of the nucleotide sequence encoding at least part of the rTCR-V into a specific genomic location. In some embodiments, the vector is directed for incorporation of the nucleotide sequence encoding at least part of the rTCR-V into the genomic TCR locus. In some embodiments, the vector is directed for incorporation of the nucleotide sequence encoding at least part of the rTCR-V into the TRAC locus. In some embodiments, the vector is directed for incorporation of the nucleotide sequence encoding at least part of the rTCR-V into the genomic TCR locus by homologous recombination.

In some embodiments, the present invention provides a host cell expressing the rTCR-V disclosed herein, or comprising the nucleic acid disclosed herein or the vector disclosed herein.

The host cell may be any host cell, depending on the purpose. In some embodiments, the host cell is used for storing the rTCR-V. In some embodiments, the host cell is used for treating a subject.

In some embodiments, the host cell is a T cell. The T cell may be any suitable type of T cell, including cells expressing CD3 (CD3⁺), CD8 (CD8⁺), CD4 (CD4⁺), and/or other relevant T cells markers. In some embodiments, T cells express at least CD3.

By expressing the rTCR-V disclosed herein, the T cell is specific for the viral antigen for which the expressed rTCR-V is specific, restricted by the HLA type of the TCR. Accordingly, when the T cell is administered to a subject, it may participate in an immune response against the viral antigen.

Importantly, the HLA restriction of the viral antigen is provided by the rTCR-V. Therefore, there is no need for the host cell to be of a specific HLA type. If needed, the endogenous TCR of the host T cell may be abolished, as discussed below.

Therefore, in some embodiments, the HLA type of the host T cells is not the predetermined HLA type.

In some embodiments, the present invention provides the composition described herein, or a composition comprising the cells or host cells described herein, further comprising a pharmaceutically acceptable carrier.

In some embodiments, the pharmaceutical composition is prepared for immediate use in a patient in need thereof. In some embodiments, the pharmaceutical composition is prepared and stored for future use.

In some embodiments, the pharmaceutically acceptable carrier is a buffer, diluent, adjuvant, excipient, or vehicle suitable for administration with the T cells of the invention. In some embodiments, the pharmaceutically acceptable carrier may be suitable for intravenous infusion. In some embodiments, the pharmaceutically acceptable carrier may be suitable as a cryoprotectant. In some exemplary embodiments, the carrier may be DMSO (for example, at about 10%). In some embodiments, the pharmaceutically acceptable carrier may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

In some embodiments, the compositions of the invention may be provided in unit vials or bags and stored frozen until use. Unit doses may include from about 5×10⁴ to about 5×10⁹ cells per ml, in a volume of from 50 to 200 ml. Each possibility is a separate embodiment.

In some embodiments, the present invention provides the composition described herein, for use in a method of treating or preventing viral infection in a subject, by administering to the subject a therapeutically effective amount of the composition.

In some embodiments, the present invention provides the composition described herein for use in a method of treating a subject infected with a virus, the method comprising administering to the subject a therapeutically effective amount of the composition. In some embodiments, the present invention provides the composition described herein for use in a method of preventing a viral infection in a subject at risk, the method comprising administering to the subject a therapeutically effective amount of the composition.

In some embodiments, the present invention provides a composition comprising T cells each expressing a rTCR-V specific for a viral antigen restricted by a predetermined HLA type, for use in a method of treating or preventing viral infection in a subject, by administering to the subject a therapeutically effective amount of the composition.

In some embodiments, the present invention provides a composition comprising T cells each expressing a recombinant virus-specific T cell receptor (rTCR-V) specific for a viral antigen restricted by a predetermined HLA type, for use in a method of treating a subject infected with a virus, the method comprising administering to the subject a therapeutically effective amount of the composition. In some embodiments, the present invention provides a composition comprising T cells each expressing a recombinant virus-specific T cell receptor (rTCR-V) specific for a viral antigen restricted by a predetermined HLA type, for use in a method of preventing a viral infection in a subject at risk, the method comprising administering to the subject a therapeutically effective amount of the composition.

In some embodiments, the present invention provides a use of the composition described herein for treating a subject infected with a virus, comprising administering to the subject a therapeutically effective amount of the composition. In some embodiments, the present invention provides a use of the composition described herein for preventing a viral infection in a subject at risk, comprising administering to the subject a therapeutically effective amount of the composition.

In some embodiments, the present invention provides a use of the composition described herein for the preparation of a medicament for treating a subject infected with a virus. In some embodiments, the present invention provides a use of the composition described herein for the preparation of a medicament for preventing a viral infection in a subject at risk.

The term “subject at risk”, or “subject at risk of being infected by a virus”, as used herein, relates to a subject who does not show symptoms of being affected with a virus, but is at risk for developing a viral disease. For example, this may be a subject who underwent organ transplantation (or some other procedure involving contact with human biological material), and it was later found that the transplantation donor was infected with a certain virus. The subject at risk may also be at risk because of a potential viral contamination in the surrounding of the subject, for example, by being in contact, or around an individual who was infected with a virus. When a subject is at risk for a known virus or viruses, administering the compositions or the VSTs of the invention may prevent a viral infection.

It is noted that the composition mentioned here with respect to its use is the same composition described herein above in more detail, and therefore all embodiments described above with respect to the composition also apply to the composition described here with respect to its use.

In some embodiments, the subject is infected with at least one virus selected from adenovirus (ADV), cytomegalovirus (CMV), BK virus (BKV), John Cunningham virus (JC), Epstein-Barr virus (EBV), human herpes virus 6 (HHV6), human immunodeficiency virus (HIV), and any combination thereof.

In some embodiments, the administration is part of an adoptive cell therapy (ACT).

In some embodiments, the composition is for use in ACT against virally infected cells.

As used herein, the terms “adoptive cell transfer therapy” or “ACT” refer to administration of ex vivo-activated and/or -expanded autologous or allogeneic viral-reactive T cells, either as is or in a suitable composition in the presence of one or more suitable excipients (such as, for example, a suitable buffer).

In some embodiments, the administration is following transplantation of an organ or cells from a transplantation donor, such as a hematopoietic stem cell transplantation (HSCT) or a solid organ transplantation (SOT) from a donor.

In some embodiments, the T cells are not derived from the subject. In some embodiments, the T cells are not derived from the transplantation donor. In some embodiments, the T cells are not derived from the subject or from the transplantation donor.

In some embodiments, the T cells are derived from the subject. In some embodiments, the T cells are derived from the transplantation donor. In some embodiments, the T cells are derived from the subject or from the transplantation donor.

In some embodiments, the T cells are autologous or allogeneic to the subject. In some embodiments, the T cells are autologous to the subject. In some embodiments, the T cells are allogeneic to the subject. In some embodiments, the T cells are HLA-matched to or haploidentical with the subject.

The terms “autologous”, “allogeneic”, and “haploidentical”, as used herein, refer to the level of identity (match) between HLA molecules of donor (or transplantation donor) cells and of recipient cells.

The major histocompatibility complex (MHC) is a collection of cell surface molecules encoded by a large number of genes in mammals, which are extremely polymorphic and therefore variable in the population. MHC molecules, also referred to in humans as human leukocyte antigens (HLA), include Class I and Class II molecules. Class I molecules are expressed on all nucleated cells and present processed peptides from within the cell mainly to cytotoxic (CD8) cells, while class II molecules are expressed on the surface of immune system cells, and present external antigens mainly to CD4 cells.

HLA molecules are important for organ transplantations, and mismatched HLA types (i.e. transplantation donor and recipient expressing different HLA molecules) are a major cause of transplanted organs rejection. Therefore, the compatibility of an organ donation is determined, inter alia, by the level of identity of HLA molecules (HLA match) between the transplantation donor and the subject. However, one of the advantages of the present invention is that there may not necessarily be a need to match the T cells to the subject by HLA typing, since the rTCR-V is already selected to match the subject's HLA type.

Nevertheless, this does not preclude using T cells matching the HLA type of the subject, if needed.

The term “autologous”, as used herein, refers to cells which are derived from the same subject and therefore have the same HLA type.

The term “allogeneic”, as used herein, refers to a donor, or transplantation donor, of the same species as the subject, but expressing HLA molecules at least to some extent different than those of the subject. Allogeneic cells may cause graft-host disease when used for cell or organ transplantation.

The term “haploidentical”, as used herein, refers to an allogeneic match, which is usually from a family member who is about 50% identical to the subject.

In some embodiments, the present invention provides a method for preparing T cells expressing a recombinant virus-specific T cell receptor (rTCR-V) specific for a viral antigen restricted by a predetermined HLA type, the method comprising the steps of:
a. providing at least one virus-specific nucleotide sequence derived from a variable region of a T cell receptor (TCR) from a T cell specific to the viral antigen restricted by the predetermined HLA type;
b. preparing a nucleic acid molecule comprising the at least one virus-specific nucleotide sequence and encoding a partial or a complete rTCR-V alpha and/or beta chain; and
c. introducing the nucleic acid molecule into host T cells, thereby obtaining T cells expressing a rTCR-V specific for a viral antigen restricted by a predetermined HLA type.

In some embodiments, the present invention provides a method for preparing a recombinant virus-specific T cell receptor (rTCR-V) specific for a viral antigen restricted by a predetermined HLA type, the method comprising the steps of:
a. providing at least one virus-specific nucleotide sequence derived from a variable region of a T cell receptor (TCR) from a T cell specific to the viral antigen restricted by the predetermined HLA type; and
b. preparing a nucleic acid molecule comprising the at least one virus-specific nucleotide sequence and encoding a partial or a complete rTCR-V alpha and/or beta chain.

The definitions and embodiments related to terms that have been mentioned herein above, such as “viral antigen” and “restricted by” are the same as detailed herein above.

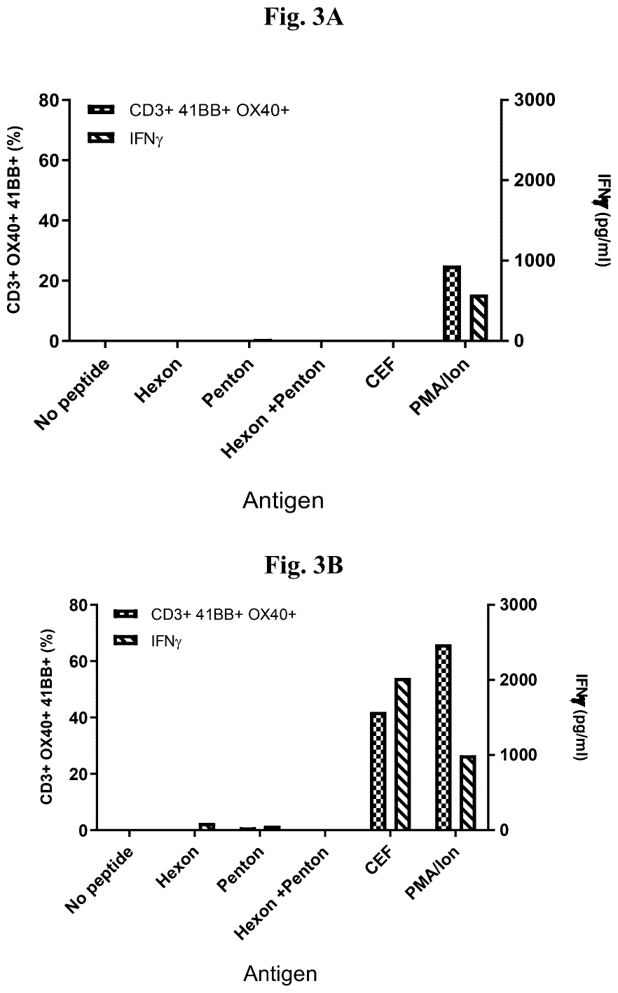

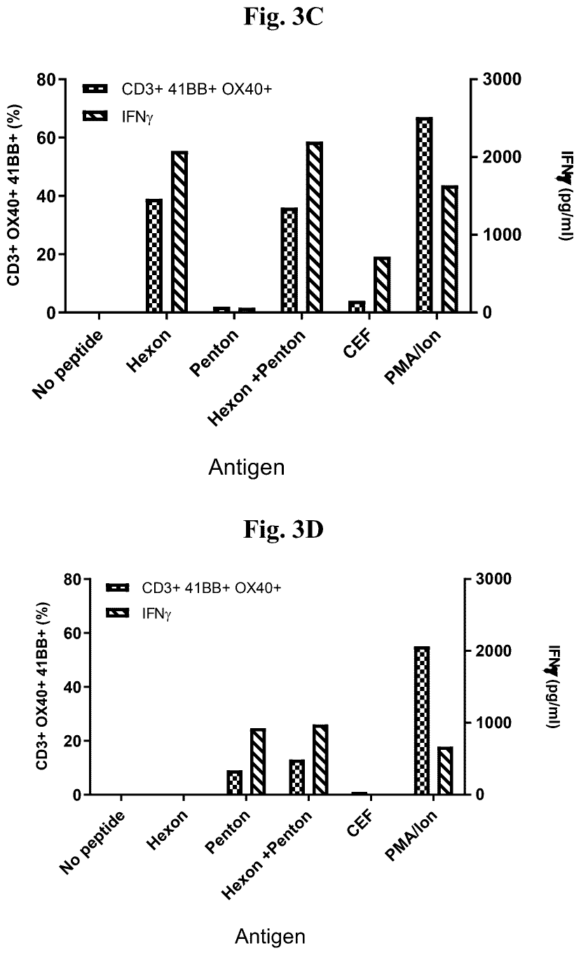

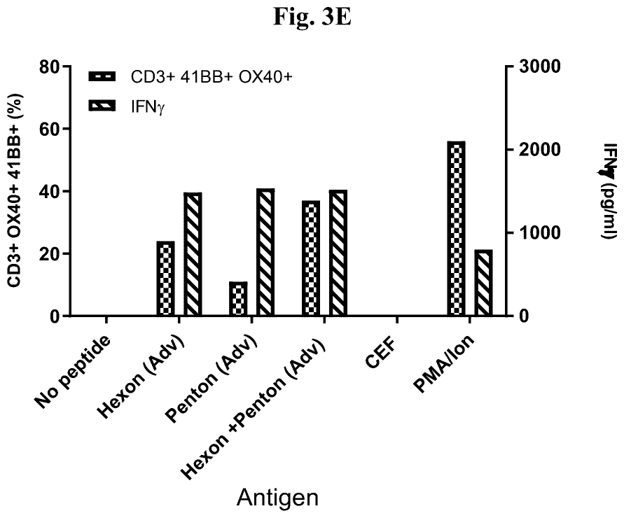

FIG. 1, discussed herein above, presents a schematic illustration of an embodiment of the method for preparing the rTCR-V T cells. FIGS. 3J-3L show the specificity and reactivity of rTCR-V T cells obtained by the method described here.

The term “virus-specific nucleotide sequence”, as used herein, relates to a nucleotide sequence which encodes at least part of the variable region of the TCR gene from the T cell specific to the viral antigen, and which determines the TCR antigen specificity. In some embodiments, the virus-specific nucleotide sequence comprises at least an alpha and/or a beta chain CDR3 sequence.

In some embodiments, the virus-specific nucleotide sequence comprises a TCR alpha and/or a beta chain CDR3 sequence. In some embodiments, the virus-specific nucleotide sequence comprises a TCR alpha chain V-J and/or a beta chain V-D-J region sequence. In some embodiments, the virus-specific nucleotide sequence comprises a TCR alpha and/or a beta chain complete variable region sequence.

The virus-specific nucleotide sequence may be retrieved by any suitable method.

In some embodiments, the virus-specific nucleotide sequence is derived from a virus-specific T cell (VST). In some embodiments, the virus-specific nucleotide sequence is derived from a naturally occurring VST. In some embodiments, the virus-specific nucleotide sequence is derived from a VST prepared by in vitro activating T cells with the viral antigen or with APCs presenting peptides derived from the viral antigen.

Accordingly, in some embodiments, providing the at least one virus-specific nucleotide sequence (as per step (a) above) is conducted by the following steps, prior to step (a) above:
1. providing precursor cells of the predetermined HLA type;
2. providing (i) at least one stimulating antigen derived from the at least one viral antigen, or (ii) antigen presenting cells (APCs) of the predetermined HLA type presenting at least one viral peptide derived from the viral antigen;
3. in vitro stimulating (IVS) the precursor cells by incubating with the APCs or with the at least one stimulating antigen of step 2 to obtain virus-specific stimulated T cells (VSTs);
4. isolating from the VSTs individual T cells specific for the viral antigen; and
5. sequencing at least part of a variable region from at least one TCR from the individual T cells specific to the viral antigen, thereby providing the at least one virus-specific nucleotide sequence.

FIGS. 3, 4, and 5A-5I demonstrate the specificity and reactivity of the VSTs obtained by the methods described here.

The term “precursor cells” as used here, relates to cells, such as donor cells, which are suitable for the invention. The precursor cells must include T cells, and therefore may be any group of cells including T cells, including, but not limited to white blood cells, peripheral blood mononuclear cells (PBMCs), mononuclear cells (MNCs), immune cells, and T cells.

The precursor cells may be obtained from a fresh or from a frozen biological sample. In some embodiments, the precursor cells are cultured cells.

In some embodiments, the precursor cells are PBMCs.

HLA typing of precursor cells and/or for APCs may be performed by any suitable method (e.g., serological or molecular tissue typing), for example based on the population repertoire (for example, see the Allele frequency net database (AFND)).

In some embodiments, the precursor cells are from a donor that is not infected with the virus from which the viral antigen is derived. In some embodiments, the donor is not infected with a virus selected from ADV, BKV, CMV, HHV6, JC, and/or HIV. Since the precursor cells are not necessarily from a transplantation donor, there may be an advantage to a donor who was already exposed to the virus, and therefore may have a higher level of T cells specific to the virus. Accordingly, in some embodiments, the precursor cells are from a donor who has previously been infected with the virus from which the viral antigen is derived.

In some embodiments, the donor is of the predetermined HLA type.

The stimulating antigens derived from the one or more viral antigens are added in order to activate and expand T cell clones which are capable of recognizing the viral antigens. Accordingly, the stimulating antigens derived from the one or more viral antigens may be provided in any suitable form for stimulating T cell clones which recognize the viral antigens.

The stimulating antigen derived from the at least one viral antigen may be the complete viral antigen, portion(s) of the viral antigen, or peptides derived from the viral antigen, that are capable of eliciting a response by T cells against the viral antigen. It is also conceivable that the stimulating antigen includes modifications or variations compared to the viral antigen.

In some embodiments, the length of the peptides is between about 10-20, about 12-18, or about 15, amino acids. In some embodiments, the peptides derived from the viral antigens include at least two peptides derived from the same viral antigen, these at least two peptides having an overlap in sequence between them. In some embodiments, the overlap in sequence is at least 2-10 amino acids. In some embodiments, the overlap in sequence is at least 2, 3, 3, 4, 6, 8, or 10 amino acids. In some embodiments, any of the amino acid sequences of the stimulating antigens may include one or more modified amino acids.

In some embodiments, the stimulating antigens are generally suitable for presentation by an HLA-A, B, or C molecular complex, and in some embodiments an HLA-A2 molecular complex. In some embodiments, the stimulating antigens are generally suitable for presentation by HLA-DR/DP/DQ (Class II MHC) HLA complexes.

In some embodiments, the stimulating antigens are pools of peptides derived from at least one viral antigen. In some embodiments, the stimulating antigens are pools of peptides derived from an ADV hexon protein. In some embodiments, the stimulating antigens are pools of peptides derived from a CMV pp65 protein. In some embodiments, the stimulating antigens are pools of peptides derived from BKV VP1 and/or LTA.

In some embodiments, the APCs are professional APCs, such as dendritic cells, B cells, or macrophages, which can present an antigen both in the context of class I HLA molecules and class II HLA molecules. In some embodiments, the APCs are any nucleated cells, which present antigens in the context of class I HLA molecules. In some embodiments, the APCs are professional APCs. In some embodiments, the APCs are dendritic cells or macrophages.

According to some embodiments, the stimulating antigens may be provided in step (2) for IVS in step (3) in either of two ways, as explained below. According to some embodiments, stimulating antigens are mixed directly with the precursor cells, and the IVS reaction is then incubated for a “direct” IVS (option i). According to some embodiments, stimulating antigens are mixed with APCs and incubated for the APCs to present peptides derived from the viral antigens. Then, the “loaded” APCs are incubated with the precursor cells for “indirect” IVS (option ii).

In some embodiments, “direct” IVS is used (option (i)) and the precursor cells comprise cells other than T cells. In some embodiments, “direct” IVS is used and the precursor cells comprise APCs (for example, if the precursor cells are PBMCs). In some embodiments, “direct” IVS is used and the precursor cells comprise professional APCs. In some embodiments, the “direct” IVS is used and the precursor cells are PBMCs.

In some embodiments, providing antigen presenting cells (APCs) of the predetermined HLA type presenting at least one viral peptide derived from the viral antigen is conducted by the following step, prior to step (2) above: obtaining APCs from a sample of a donor having the predetermined HLA type; and incubating the APCs with the stimulating antigens derived from the viral antigen; thereby obtaining APCs presenting at least one viral peptide derived from the viral antigen.

In some embodiments, the APCs and the precursor cells are from the same source, such as from the same donor.

In some embodiments, the incubating the IVS reaction in step (3) above is conducted in the presence of IL2. In some embodiments, the incubating the IVS reaction in step (3) above is conducted in the presence of IL2 at a final concentration of at least 100 IU/ml, at least 200 IU/ml, about 100 IU/ml-500 IU/ml, about 200 IU/ml-400 IU/ml, or about 300 IU/ml of added IL2.

In some embodiments, the IVS reaction in step (3) above may be incubated in any suitable medium for culturing monocytes or T cells, such as RPMI, AIM-V™, or the VST medium described in the examples.

In some embodiments, the incubating of the IVS reaction in step (3) is conducted for a length of about 8-15 days, or about 10-12 days.

In some embodiments, isolating individual T cells specific for the viral antigen (VSTs) is conducted by the following steps: reactivating the stimulated T cells with stimulating antigens derived from the viral antigen or with APCs presenting viral peptides derived from the viral antigen; detecting and isolating VSTs by their expression of activation markers; and plating each isolated T cell individually; thereby obtaining individual VSTs.

In some embodiments, the activation markers are 4-1BB and/or OX40.

After individual VSTs are obtained, their TCR is isolated and sequenced. The sequences of the variable regions of the alpha and the beta chains, or the CDR3, which determines the TCR specificity, are used in step (a) of the above method. Some nonlimiting examples of such sequences may be found in Table 2.

In step (b) of the method, the virus-specific nucleotide sequence provided is used as a basis for preparing a nucleic acid molecule which encodes a rTCR-V including the virus-specific sequence in its variable region.

The nucleic acid molecule prepared in step (b) may encode a complete rTCR-V, or a partial rTCR-V. The term “a partial rTCR-V” relates to any portion of the rTCR-V that includes at least the virus-specific sequence provided in step (a), comprising at least a CDR3 sequence.

In some embodiments, the partial rTCR-V is an alpha chain and/or a beta chain of the rTCR-V. In some embodiments, the partial rTCR-V is an alpha chain and/or a beta chain variable region of the rTCR-V. In some embodiments, the partial rTCR-V is an alpha chain and/or a beta chain CDR3 region of the rTCR-V.

In some embodiments, the virus-specific sequence, which comprises at least a CDR3 region of a TCR alpha and/or beta chain, is reconstructed into a sequence encoding a partial or a complete TCR (rTCR-V) encoding sequence by adding TCR encoding sequences. Since the CDR3 determines the antigen specificity, generally any TCR may be a source for the added TCR sequences. In some embodiments, the added TCR sequences are from a single source. In some embodiments, the added TCR sequences are from more than one source. In some embodiments, the added TCR sequences are from a human source. In some embodiments, the added TCR sequences are from a mouse source. In some embodiments, the added TCR sequences comprise both human and mouse sequences. In some embodiments, the added TCR sequences comprise only constant region sequences (such as alpha chain and/or beta chain constant region sequences). In some embodiments, the added TCR sequences comprise framework sequences.

The nucleic acid molecule may be constructed by any suitable method. For example, in some embodiments, the complete rTCR-V sequence is synthesized. In some embodiments, the rTCR-V sequence is prepared by fusing (e.g., by ligating) nucleic acid molecules comprising the elements, such as the virus-specific sequence and the rest of the TCR sequences. In some embodiments, the rTCR-V sequence is prepared by a combination of nucleic acid synthesis and ligation to existing/cloned sequences. For example, in some embodiments, the virus-specific sequence is synthesized and ligated to already cloned TCR sequences.

The nucleic acid molecule encoding the partial or complete rTCR-V may be provided in any way suitable, e.g., as a free molecule or ligated to a suitable vector, for storage, or for incorporating into T cells. In some embodiments, the nucleic acid encoding the partial or complete rTCR-V is cloned into a retroviral vector such as MSGV1.

In step (c), the nucleic acid molecule is introduced into host T cells.

The T cell may be any suitable type of T cell, including cells expressing CD3 (CD3⁺), CD8 (CD8⁺), CD4 (CD4⁺), and/or other relevant T cells markers. In some embodiments, T cells express at least CD3.

By expressing the rTCR-V disclosed herein, the T cell is specific for the viral antigen for which the expressed rTCR-V is specific, restricted by the HLA type of the rTCR-V. Accordingly, when the T cell is administered to a subject, it may participate in an immune response against the viral antigen.

Importantly, the HLA restriction of the viral antigen is provided by the rTCR-V. Therefore, there is no need for the host cell to be of a specific HLA type. If needed, the endogenous TCR of the host T cell may be abolished, as discussed below.

Therefore, in some embodiments, the HLA type of the host T cells is not the predetermined HLA type.

When the nucleic acid molecule comprises sequences encoding the complete rTCR-V molecule, it does not need to be directed to the TCR locus and may have a promoter as part of the vector. However, it may still be directed to the TCR locus, and may be used to replace the endogenous TCR (e.g., by knock-in, such as by homologous recombination). On the other hand, if the nucleic acid molecule encodes for a partial rTCR-V sequence, it may be used by replacing the respective part of the endogenous TCR, e.g., by homologous recombination.

In some embodiments, the nucleic acid molecule encodes the complete rTCR-V and the vector is not directed for incorporation of the rTCR-V encoding sequence into a specific genomic region. In some embodiment, the vector comprises a promotor for driving expression of the rTCR-V.

In some embodiments, the vector is directed for incorporation of the rTCR-V encoding sequence into a specific genomic location. In some embodiments, the vector is directed for incorporation of the rTCR-V encoding sequence into the genomic TCR locus. In some embodiments, the vector is directed for incorporation of the rTCR-V encoding sequence into the TRAC locus. In some embodiments, the vector is directed for incorporation of the rTCR-V encoding sequence into the genomic TCR locus by homologous recombination.

Introducing of the nucleic acid molecule, or the vector comprising it, into the host T cells may be carried out by any method known in the art, such as electroporation, transduction, and virus-free systems using CRISPR genome editing by HDR, etc, depending on the vector and on the purpose.

In some embodiments, the host T cells are activated prior to introducing the nucleic acid molecule. In some embodiments, the host T cells are not activated prior to introducing the nucleic acid molecule.

In some embodiments, the method further comprises a step of knocking-out the endogenous TCR of host T cells. This step may be carried out before or after introducing the nucleic acid molecule into the host cells in step (c) above.

In some embodiments, the step of knocking-out the endogenous TCR is carried out by a CRISPR-cas9 system or by a similar system such as zinc finger nucleases (ZFNs), Transcription activator-like effector nuclease (TALEN), or megaTAL nucleases, or by base and prime editing. In some embodiments, the knock-out is directed to the TRAC locus. In some embodiments, the knock-out is conducted by electroporation of an RNP complex containing CRISPR-cas9 and a gRNA directed to the TRAC locus. In some embodiments, a high fidelity Cas9 protein such as HiFi Cas9 is used (Schmid-Burgk et al. 2020, Highly Parallel Profiling of Cas9 Variant Specificity. Mol Cell. 78(4):794-800 e8).

In some embodiments, the present invention provides a method for preparing T cells expressing a recombinant virus-specific T cell receptor (rTCR-V) specific for a viral antigen restricted by a predetermined HLA type, the method comprising the steps of:
a. providing precursor cells of the predetermined HLA type;
b. providing (i) at least one stimulating antigen derived from the at least one viral antigen, or (ii) antigen presenting cells (APCs) of the predetermined HLA type presenting at least one viral peptide derived from the viral antigen;
c. in vitro stimulating (IVS) the precursor cells by incubating with the APCs or with the at least one stimulating antigen of step (b) to obtain virus-specific stimulated T cells (VSTs);
d. isolating from the VSTs individual T cells specific for the viral antigen;
e. sequencing at least part of a variable region from at least one TCR from the individual T cells specific to the viral antigen, thereby providing the at least one virus-specific nucleotide sequence;
f. preparing a nucleic acid molecule comprising the at least one virus-specific nucleotide sequence and encoding a partial or a complete rTCR-V alpha and/or beta chain; and
g. introducing the nucleic acid molecule into host T cells, thereby obtaining T cells expressing a rTCR-V specific for a viral antigen restricted by a predetermined HLA type.

In some embodiments, there are provided T cells expressing a recombinant virus-specific T cell receptor (rTCR-V) polypeptide specific for a viral antigen restricted by a predetermined HLA type, prepared by the methods disclosed herein.

These T cells may be either stored, e.g., by cryopreservation, or used for treating a subject in need, such as a subject infected by a virus, as described herein.

In some embodiments, the cells are cryopreserved.

In some embodiments, the HLA type of the T cells is not the predetermined HLA type.

In some embodiments, there is provided a method of treating a viral disease in a subject, the method comprising administering to the subject a therapeutically effective amount of the composition or of the T cells disclosed herein.

In some embodiments, there is provided a method of preventing a viral disease in a subject at risk, the method comprising administering to the subject a therapeutically effective amount of the composition or of the T cells disclosed herein.

It is noted that since the terms “composition described herein” or “T cells described herein”, reference the compositions and T cells of the invention as described herein above in more detail, all embodiments described above with respect to these terms also apply to the respective term mentioned here with respect to the methods of treatment.

In some embodiments, the viral disease is caused by a virus selected from: adenovirus (ADV), cytomegalovirus (CMV), BK virus (BKV), John Cunningham virus (JC), Epstein-Barr virus (EBV), human herpes virus 6 (HHV6), and human immunodeficiency virus (HIV).

In some embodiments, the administration is following transplantation of an organ or cells from a transplantation donor, such as a hematopoietic stem cell transplantation (HSCT) or a solid organ transplantation (SOT).

In some embodiments, the administration is part of an adoptive cell therapy (ACT).

In some embodiments, the composition is for use in ACT against virally infected cells.

In some embodiments, the T cells are autologous or allogeneic to the subject.

In some embodiments, the T cells are autologous to the subject. In some embodiments, the T cells are allogeneic to the subject. In some embodiments, the T cells are HLA-matched to or haploidentical with the subject. In some embodiments, the T cells are not HLA-matched to the subject.

The administration of the T cells and/or compositions comprising them may be conducted by any suitable method, such as, but not limited to intravenous or subcutaneous infusion, such as bolus infusion, guiding infusion, periocular infusion, subretinal infusion, intravitreal infusion, transmural infusion, coarctation infusion, Intravenous infusion, sub-conjunctival injection, subconjunctival injection, intrathoracic injection, posterior infusion, periocular infusion, or hindlimb transmission.

In some embodiments, the compositions may be administered by parenteral, intrapulmonary, or intranasal administration. Parenteral infusions include intramuscular, intravenous, intraarterial, intraperitoneal, intrathecal, intracranial, or subcutaneous administration.

In some embodiments, the administration is an intravenous administration. In some embodiments, the administration is by an intravenous injection or infusion.

In some embodiments, the administration is a single administration. In some embodiments, the administration comprises multiple administrations. In some embodiments, the administration comprises administration for a day or more than one day, e.g., up to 1 day, 2 days, or 3 days. In some embodiments, the administration may be repeated as needed, e.g., when viral load of the patient increases.

In some embodiments, the compositions of the invention are administered in combination with an additional therapeutic agent. In some embodiments, the therapeutic agent is an antirejection medicine or an antibiotic (such as an antiviral, antibacterial, or antifungal) agent. In some embodiments, the therapeutic agent is selected from a steroid, such as prednisone or an equivalent, a kinase inhibitor, e.g., a Janus kinase inhibitor such as ruxolitinib, an antirejection medicine such as mycophenolate mofetil, and an antiviral agent such as ganciclovir.

The term “treating”, as used herein, refers to means of obtaining a desired physiological effect, in this case, partially or completely curing the infection and/or symptoms thereof. The term may relate to ameliorating or inhibiting the infection, i.e. arresting its development or curing it completely by eradicating the virus.

The term “preventing”, as used herein, refers to causing the viral infection or symptoms thereof not to appear in the subject, or delaying the onset of the viral infection or symptoms thereof, such that they do not appear at the time they are expected to appear in similar cases, or causing the viral infection or symptoms thereof to appear at a diminished level.

The term “therapeutically effective amount” as used herein means an amount of the composition or of the VSTs that will result in a suitable amelioration or eradication of the viral injection. The amount must be effective to achieve the desired therapeutic effect as described above, depending inter alia on the type and severity of the infection, and the treatment regime. The therapeutically effective amount is typically determined in appropriately designed clinical trials (dose range studies) and the person skilled in the art will know how to properly conduct such trials to determine the effective amount. As generally known, an effective amount depends on a variety of factors including the affinity of the ligand to the receptor, its distribution profile within the body, a variety of pharmacological parameters such as half-life in the body, on undesired side effects, if any, and on factors such as age and gender, etc.

The present invention also provides a comprehensive library of pathogen-specific, and specifically virus specific TCRs (rTCR-Vs) and/or of cells including them, that can serve both for research evaluation of immunogenicity against each pathogen protein and for clinical applications such as treating infections, especially following transplantations.

Advantageously, the library is intended to provide a high quality and quantity of antigen-specific T cells for a variety of antigens restricted by various HLA types, so that they may be available for patients in need without the need to look for a suitable donor or worry about GvHD.

In some embodiments, there is provided a library comprising a plurality of rTCR-Vs and/or of T cells comprising them, each as defined herein, wherein the library comprises at least two rTCR-V (or T cells) which are specific for viral antigens derived from the same virus, but the viral antigens are restricted by a different HLA type in each of the two rTCR-Vs.

It is noted that the T cells and TCRs described herein as part of the library are described above in more detail. Accordingly, all embodiments described above with respect to the T cells and TCRs of the invention also apply to the compositions which form part of the library.

The library is intended to be an “off-the shelf” library, or a biobank, which comprises rTCR-Vs and/or T cells comprising them, prepared against viral antigens from different sources, and with precursor cells from different individuals in order to provide various HLA-type restrictions. Accordingly, the library comprise rTCR-Vs and/or T cells comprising them against antigens derived from various different viruses, such as ADV, CMV, BKV, JC, HHV6, and/or HIV. Further, the library also comprises rTCR-Vs and/or T cells comprising them against antigens from the same viruses, but in a different HLA background, since the rTCR-Vs and/or T cells comprising them are from a variety of donors. This way, the library is able to provide allogeneic T cells for treating a viral infection in a subject, by providing VSTs specific to the virus, and also matching the HLA type of the subject.

As explained above, this is achieved by using precursor cells from a variety of individuals (donors) having a variety of HLA types, and challenging each precursor cell with viral antigens from a variety of viruses, so as to obtain a variety of rTCR-Vs and/or T cells comprising them with specificities covering a range of viruses, on different HLA backgrounds.

The compositions comprising the library may be prepared by methods described herein.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

TABLE 1
Reagents

| Reagent | Catalog | manufacturer/Distributer |
|---|---|---|
| Ficoll-Paque ® PREMIUM | 95021-207 | GE healthcare/Daniel biotech |
| RPMI1640 | 01-104-1A | Biological industries |
| AIM-V ™ medium (Research | 12055091 | Gibco/Rhenium |
| grade) | | |
| human serum | H-4522-10ML | Sigma |
| L-Glutamine | 03-020-1B | Biological industries |
| Pen/Strep (penicillin, 10⁴ | 03-031-1B | Biological industries |
| U/ml, streptomycin, 10⁴ | | |
| μg/ml) | | |
| Recombinant human IL2 | CH1001 | Megapharm, Chiron Novartis, or |
| | | Proleukin ® (aldesleukin) (drug) |
| LTA (BKV) Peptide library | 170-076-139 | Miltenyi/Almog |
| (clinical grade) | | |
| VP1 (BKV) Peptide library | 170-076-138 | Miltenyi/Almog |
| (clinical grade) | | |
| Hexon (ADV) peptide library | 170-076-106 | Miltenyi/Almog |
| (clinical grade) | | |
| Penton (ADV) library | | Miltenyi/Almog |
| pp65 (CMV) peptide library | 170-076-109 | Miltenyi/Almog |
| (clinical grade) | | |

### Example 1: in Vitro Stimulation and Enrichment for Pathogen-Specific T Cells

In order to stimulate and enrich for pathogen-specific T cells from healthy donors, PBMCs were isolated from a healthy donor and monocyte-derived dendritic cells were produced to serve as antigen-presenting cells (APCs).

The produced APCs were loaded with a commercial CEF T cell stimulation pool (CEFX™ JPT Peptide Technologies) composed of 175 known HLA class I epitopes derived from both bacteria and viruses, including cytomegalovirus (CMV), Epstein-Barr virus (EBV), and influenza virus. PBMCs were in vitro stimulated (IVS) with CEF-loaded APCs for seven days in the presence of IL2 (IVS 1).

The cells were then restimulated (IVS 2) for seven more days. To test for the level of CEF-specific T cells, each IVS sample was co-cultured with APCs loaded with CEF and the expression of the T cell activation marker 4-1BB on the surface of CD8 T cells was measured. APCs without CEF and non-activated PBMC were served as controls.

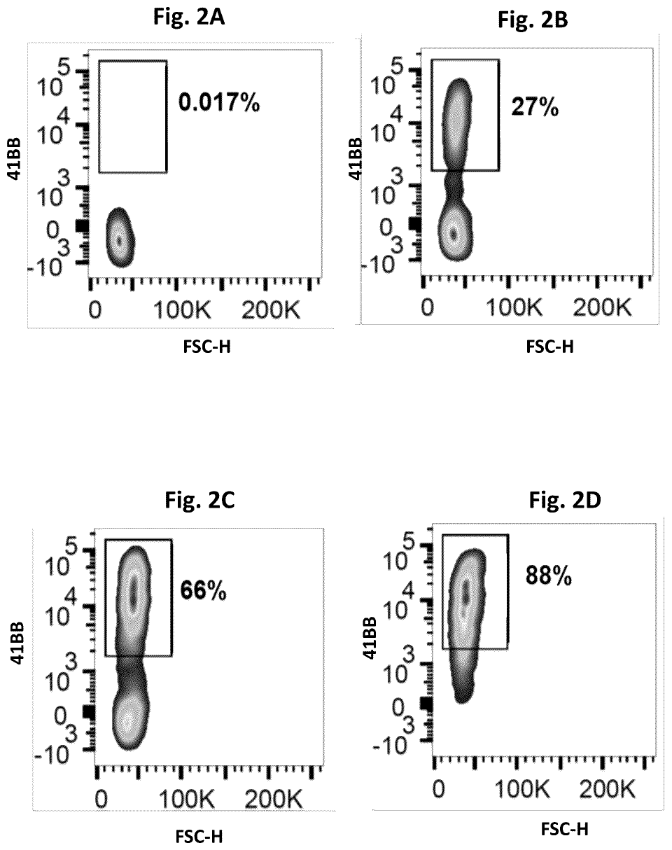

As can be seen in the results presented in FIG. 2, in the initial PBMC culture, 27% of the cells were CEF specific (FIG. 2B), and following IVS1, the level of specific cells increased to 66% (FIG. 2C). Additional enrichment was observed following the second stimulation IVS2 (88%) (FIG. 2D). The results presented in FIG. 2 clearly show the ability to stimulate and enrich pathogen-specific T cells.

### Example 2: In vitro Stimulation (IVS) with Peptides of Adenovirus (ADV) or Cytomegalovirus (CMV)

Cells were prepared from 30 ml of a donor blood sample from a. Blood samples were diluted two-fold with MACS® buffer, and PBMCs were isolated on a Ficoll® gradient, and resuspended in PBS. PBMCs were then centrifuged and resuspended in VST medium containing 2 mM L-glutamine, 5% human serum in AIM-V™ medium, plated in 24 well plates at 2×10⁶ PBMCs per 250 μl medium, and incubated at 37° C., 5% CO2 for 2 hours.

For preparing antigen-presenting APCs, one hour before the incubation ends, the desired peptides were prepared at 250 μl/per sample to a final concentration of 1 μg/ml, and were added to the plates in triplicates.

For obtaining T cells, 10×10⁶ PBMCs in VST medium from the same sample were separated by using magnetic beads (e.g., by Pan T Cell MicroBeads, (Miltenyi Biotech)), according to the manufacturer's instructions.

For IVS, 2×10⁶ T cells were added to the APCs in each well in a final volume of 1 ml, and the cells were incubated at 37° C., 5% CO₂ for 72 hours. Cells were then fed by adding 1 ml of CTL media with IL2 (600 IU/ml) to each well (to a final concentration of 300 IU/ml) and incubating at 37° C. for 72 h. Cells were further fed and split as needed. Cells were incubated during IVS with all peptides for 10-12 days to enrich for peptide-specific T cells.

To test for the level of ADV-specific T cells, each IVS sample was co-cultured with APCs loaded with ADV peptide libraries penton or hexon, or CEF control peptides, and the expression of the T cell activation markers OX40 and 4-1BB on the surface of T cells were measured by fluorescence cell sorter (FACS), and the level presented in FIGS. 3A-3E demonstrate that the IVS enriched for ADV specific T cells to a level of 20-60% of the T cell culture. Furthermore, the enriched ADV-specific T cells showed specificity to both the hexon and penton peptides and not to the unloaded or CEF controls. CEF-loaded PBMCs were used as a positive control, and unloaded cells served as a negative control.

### Example 3: IVS Against ADV, BKV, and CMV

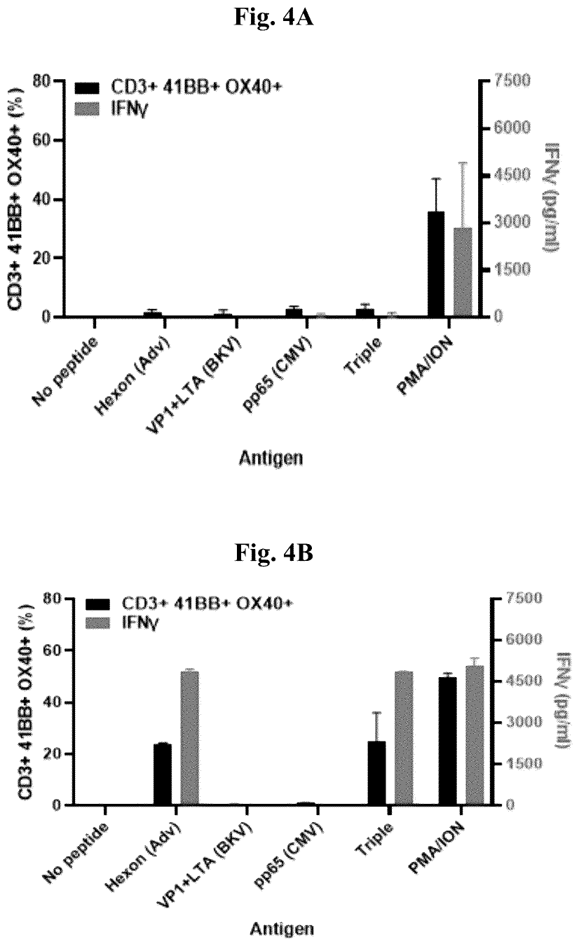

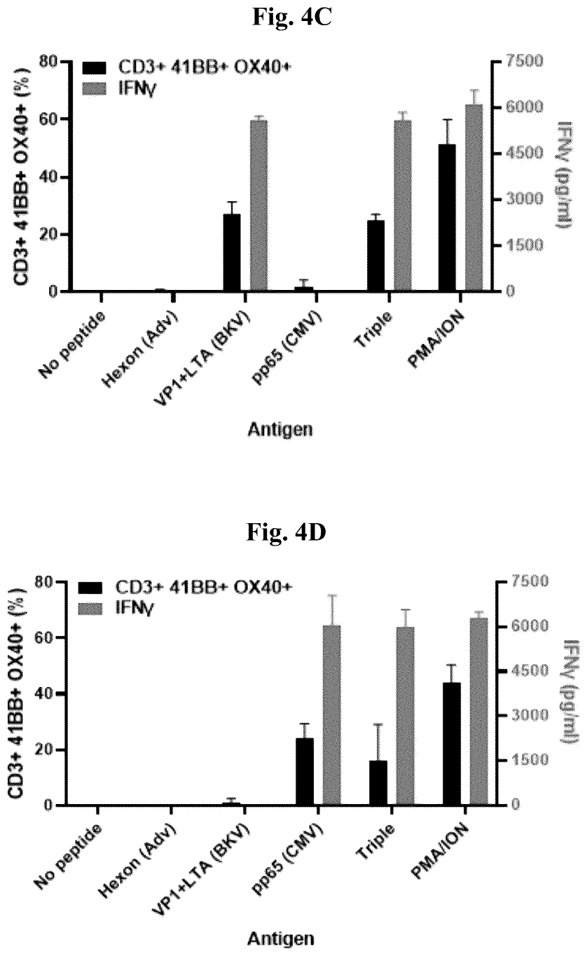

PBMCS went through IVS for 11 days with APCs loaded with viral peptide pools including peptides from adenovirus (ADV, capsid protein hexon peptides), BK virus (BKV, capsid protein VP1, and large T-antigen LTA peptides), and cytomegalovirus (CMV, structural protein pp65), as described above. Unloaded APCs served as negative controls. The virus-specific T cells (VSTs) resulting from each IVS were restimulated with each peptide pool. PMA/ION was used as positive control. Restimulation was followed by staining for CD3 and for activation markers 4-1BB and OX40 and analyzing by FACS, and by analyzing the supernatant for IFNγ level by ELISA. The specificity of the response is clear from FIGS. 4A-4D since only when antigens match there was a response. FIG. 4B shows response to adenovirus peptides (Hexon library) only by PBMCs who went through IVS with hexon library (alone or part of the triple IVS); FIG. 4C shows response to BKV VP1+LTA peptides only by PBMCs who went through IVS with VP1+LTA BKV peptides; and FIG. 4D shows response to CMV peptide (pp65) only by PBMCs who went through IVS with CMV peptide (pp65).

### Example 4: TCR Reconstruction Based on Sequences from Reactive Clones

PBMC were in vitro stimulated with APCs loaded with the hexon (ADV), pp65 (CMV), or LTA/VP1 (BKV) peptide pools, as described above. Cells were then restimulated with the respective antigen pool and sorted for activated cells based on 4-11B1 and OX40 expression. Isolated activated T cells were then diluted and plated as single cells in 96-well, grown for 2-3 weeks in the 96 well plates and then each well was restimulated with the relevant peptide libraries. T-cell clones that were reactive against the respective peptide pool based on 41BB and OX40 upregulation (ADV: FIG. 5A-5D, CMV: FIG. 5E-5H) were sent to TCR sequencing. FIG. 5I shows IFNγ production of the positive clones (ADV: left panel, CMV: right panel). The unique TCR CDR3 sequences obtained from reactive T-cell clones are summarized in Tables 2, 3 below, which presents for each reactive TCR (all from CD4⁺ T cells) the target antigen (CMV PP65, ADV-Hexon, or a minimal CMV epitope of SEQ ID NO: 22, NLVPMVATV), HLA restriction, V, D, and J sequences of the TCR chain, and the sequence of the CDR3, which renders the specificity to antigen.

TABLE 2
List of sequences

| SEQ ID | sequence |
|---|---|
| 1 | CATDRGGGSEKLVE |
| 2 | CASSQLAGHEAFF |
| 3 | CAVWPYQAGTALIF |
| 4 | CASSQDIGAGNTEAFF |
| 5 | CASNRDDKIIF |
| 6 | CASRRQGTVYEQYV |
| 7 | METLLGVSLVILWLQLARVNSQQGEEDPQALSIQEGENATMNCSYKTSINNLQW |
| | YRQNSGRGLVHLILIRSNEREKHSGRLRVTLDTSKKSSSLLITASRAADTASYF |
| | CATDRGGGSEKLVFGKGTKLTVNP |
| 8 | MACRLLCCVVFCLLQAGPLDTAVSQTPKYLVTQMGNDKSIKCEQNLGHDTMYWY |
| | KQDSKKFLKIMFSYNNKELIINETVPNRFSPKSPDKAHLNLHINSLELGDSAVY |
| | FCASSQLAGHEAFFGQGTRLTVV |
| 9 | MLLELIPLLGIHFVLRTARAQSVTQPDIHITVSEGASLELRCNYSYGATPYLFW |
| | YVQSPGQGLQLLLKYFSGDTLVQGIKGFEAEFKRSQSSFNLRKPSVHWSDAAEY |
| | FCAVWPYQAGTALIFGKGTTLSVSS |
| 10 | MACRLLCCAVLCLLGAGELVPMETGVTQTPRHLVMGMTNKKSLKCEQHLGHNAM |
| | YWYKQSAKKPLELMFVYSLEERVENNSVPSRESPECPNSSHLFLHLHTLQPEDS |
| | ALYLCASSQDIGAGNTEAFFGQGTRLTVV |
| 11 | MEKNPLAAPLLILWFHLDCVSSILNVEQSPQSLHVQEGDSTNFTCSFPSSNFYA |
| | LHWYRWETAKSPEALFVMTLNGDEKKKGRISATLNTKEGYSYLYIKGSQPEDSA |
| | TYLCASNRDDKIIFGKGTRLHILP |
| 12 | MAIRLLCRVAFCFLAVGLVDVKVTQSSRYLVKRTGEKVFLECVQDMDHENMFWY |
| | RQDPGLGLRLIYFSYDVKMKEKGDIPEGYSVSREKKERFSLILESASTNQTSMY |
| | LCASRRQGTVYEQYVGPGTRLTVT |
| 13 | ATGGAGACACTGCTGGGCGTGAGCCTGGTCATCCTGTGGCTGCAGCTGGCCCGG |
| | GTGAACAGCCAGCAGGGAGAGGAGGACCCCCAGGCCCTGTCCATCCAGGAGGGC |
| | GAGAACGCCACCATGAATTGCAGCTACAAGACATCCATCAACAATCTGCAGTGG |
| | TATCGGCAGAACTCTGGCAGAGGCCTGGTGCACCTGATCCTGATCAGGTCTAAT |
| | GAGCGCGAGAAGCACAGCGGCCGGCTGAGAGTGACCCTGGATACAAGCAAGAAG |
| | AGCAGCAGCCTGCTGATCACCGCCTCCAGGGCCGCAGACACAGCCTCTTACTTC |
| | TGTGCAACCGATAGGGGAGGAGGCTCCGAGAAGCTGGTGTTTGGCAAGGGCACC |
| | AAGCTGACAGTGAATCCT |
| 14 | ATGGCCTGCCGGCTGCTGTGCTGCGTGGTGTTCTGCCTGCTGCAGGCCGGCCCA |
| | CTGGACACCGCCGTGTCTCAGACACCCAAGTACCTGGTGACCCAGATGGGCAAC |
| | GATAAGTCCATCAAGTGCGAGCAGAATCTGGGCCACGACACAATGTACTGGTAT |
| | AAGCAGGATAGCAAGAAGTTCCTGAAGATCATGTTTTCCTATAACAATAAGGAG |
| | CTGATCATCAACGAGACCGTGCCCAATCGGTTCTCTCCCAAGAGCCCTGACAAG |
| | GCCCACCTGAACCTGCACATCAATTCCCTGGAGCTGGGCGATTCTGCCGTGTAC |
| | TTTTGTGCCAGCTCCCAGCTGGCCGGACACGAGGCCTTCTTTGGACAGGGCACC |
| | AGACTGACAGTGGTG |
| 15 | ATGCTGCTGGAGCTGATCCCTCTGCTGGGCATCCACTTCGTGCTGAGGACCGCC |
| | AGAGCCCAGTCCGTGACACAGCCAGACATCCACATCACCGTGTCCGAGGGAGCC |
| | TCTCTGGAGCTGAGGTGCAACTACTCTTATGGCGCCACACCCTACCTGTTCTGG |
| | TACGTGCAGAGCCCTGGACAGGGACTGCAGCTGCTGCTGAAGTACTTTTCCGGC |
| | GACACCCTGGTGCAGGGCATCAAGGGCTTCGAGGCCGAGTTTAAGAGGTCTCAG |
| | AGCAGCTTCAACCTGCGCAAGCCAAGCGTGCACTGGTCCGATGCCGCCGAGTAC |
| | TTCTGTGCCGTGTGGCCATATCAGGCAGGCACAGCCCTGATCTTTGGCAAGGGC |
| | ACCACACTGAGCGTGTCTAGC |
| 16 | ATGGCCTGCAGGCTGCTGTGCTGTGCCGTGCTGTGCCTGCTGGGAGCAGGAGAG |
| | CTGGTGCCAATGGAGACCGGAGTGACCCAGACACCAAGGCACCTGGTCATGGGC |
| | ATGACAAACAAGAAGAGCCTGAAGTGCGAGCAGCACCTGGGCCACAATGCCATG |
| | TACTGGTATAAGCAGTCCGCCAAGAAGCCTCTGGAGCTGATGTTCGTGTACTCT |
| | CTGGAGGAGCGGGTGGAGAACAATAGCGTGCCCAGCCGGTTCAGCCCAGAGTGC |
| | CCTAACAGCTCCCACCTGTTTCTGCACCTGCACACCCTGCAGCCAGAGGACTCC |
| | GCCCTGTACCTGTGCGCCTCTAGCCAGGACATCGGAGCAGGCAATACAGAGGCC |
| | TTCTTTGGCCAGGGCACCCGGCTGACAGTGGT |
| 17 | ATGGAGAAGAACCCCCTGGCCGCACCTCTGCTGATCCTGTGGTTCCACCTGGAC |
| | TGCGTGAGCAGCATCCTGAATGTGGAGCAGTCCCCACAGTCTCTGCACGTGCAG |
| | GAGGGCGATTCTACCAACTTCACATGTAGCTTTCCCTCTAGCAATTTCTATGCC |
| | CTGCACTGGTACAGGTGGGAGACCGCAAAGAGCCCTGAGGCCCTGTTTGTGATG |
| | ACACTGAACGGCGACGAGAAGAAGAAGGGCCGGATCTCCGCCACCCTGAATACA |
| | AAGGAGGGCTACTCTTATCTGTACATCAAGGGCAGCCAGCCAGAGGATTCCGCC |
| | ACCTATCTGTGCGCCTCCAACCGGGACGATAAGATCATCTTTGGCAAGGGCACA |
| | AGACTGCACATCCTGCCC |
| 18 | ATGGCCATCAGGCTGCTGTGCCGCGTGGCCTTCTGTTTTCTGGCCGTGGGCCTG |
| | GTGGACGTGAAGGTGACCCAGAGCAGCAGATACCTGGTGAAGAGAACAGGCGAG |
| | AAGGTGTTCCTGGAGTGCGTGCAGGACATGGATCACGAGAACATGTTTTGGTAT |
| | CGGCAGGACCCCGGACTGGGACTGAGGCTGATCTACTTCTCTTATGACGTGAAG |
| | ATGAAGGAGAAGGGCGACATCCCTGAGGGCTACTCTGTGAGCAGGGAGAAGAAG |
| | GAGCGGTTCAGCCTGATCCTGGAGTCCGCCTCTACCAATCAGACAAGCATGTAC |
| | CTGTGCGCCTCCCGGAGACAGGGCACCGTGTACGAGCAGTATGTGGGACCCGGC |
| | ACAAGACTGACCGTGACA |
| 19 | ATGGCCTGCCGGCTGCTGTGCTGCGTGGTGTTCTGCCTGCTGCAGGCCGGCCCA |
| | CTGGACACCGCCGTGTCTCAGACACCCAAGTACCTGGTGACCCAGATGGGCAAC |
| | GATAAGTCCATCAAGTGCGAGCAGAATCTGGGCCACGACACAATGTACTGGTAT |
| | AAGCAGGATAGCAAGAAGTTCCTGAAGATCATGTTTTCCTATAACAATAAGGAG |
| | CTGATCATCAACGAGACCGTGCCCAATCGGTTCTCTCCCAAGAGCCCTGACAAG |
| | GCCCACCTGAACCTGCACATCAATTCCCTGGAGCTGGGCGATTCTGCCGTGTAC |
| | TTTTGTGCCAGCTCCCAGCTGGCCGGACACGAGGCCTTCTTTGGACAGGGCACC |
| | AGACTGACAGTGGTGGAGGACCTGCGCAACGTGACACCCCCTAAGGTGTCTCTG |
| | TTCGAGCCTAGCAAGGCCGAGATCGCCAATAAGCAGAAGGCCACCCTGGTGTGC |
| | CTGGCCAGAGGCTTCTTTCCAGATCACGTGGAGCTGTCCTGGTGGGTGAACGGC |
| | AAGGAGGTGCACTCTGGCGTGTGCACAGACCCCCAGGCCTACAAGGAGAGCAAT |
| | TACTCCTATTGTCTGTCTAGCCGGCTGAGAGTGTCCGCCACCTTTTGGCACAAC |
| | CCACGGAATCACTTCAGATGCCAGGTGCAGTTTCACGGCCTGTCTGAGGAGGAT |
| | AAGTGGCCAGAGGGAAGCCCAAAGCCAGTGACCCAGAACATCTCCGCCGAGGCA |
| | TGGGGAAGGGCAGACTGTGGAATCACATCCGCCTCTTATCAGCAGGGCGTGCTG |
| | AGCGCCACCATCCTGTACGAGATCCTGCTGGGCAAGGCCACACTGTATGCCGTG |
| | CTGGTGTCCACCCTGGTGGTCATGGCTATGGTGAAGCGCAAGAACTCTAGGGCA |
| | AAGCGCAGCGGATCCGGAGCAACCAATTTCAGCCTGCTGAAGCAGGCAGGCGAT |
| | GTGGAGGAGAACCCTGGACCAATGGAGACACTGCTGGGCGTGAGCCTGGTCATC |
| | CTGTGGCTGCAGCTGGCCCGGGTGAACAGCCAGCAGGGAGAGGAGGACCCCCAG |
| | GCCCTGTCCATCCAGGAGGGCGAGAACGCCACCATGAATTGCAGCTACAAGACA |
| | TCCATCAACAATCTGCAGTGGTATCGGCAGAACTCTGGCAGAGGCCTGGTGCAC |
| | CTGATCCTGATCAGGTCTAATGAGCGCGAGAAGCACAGCGGCCGGCTGAGAGTG |
| | ACCCTGGATACAAGCAAGAAGAGCAGCAGCCTGCTGATCACCGCCTCCAGGGCC |
| | GCAGACACAGCCTCTTACTTCTGTGCAACCGATAGGGGAGGAGGCTCCGAGAAG |
| | CTGGTGTTTGGCAAGGGCACCAAGCTGACAGTGAATCCTAACATCCAGAATCCC |
| | GAGCCTGCCGTGTATCAGCTGAAGGACCCAAGATCTCAGGATAGCACACTGTGC |
| | CTGTTCACCGACTTTGATAGCCAGATCAACGTGCCCAAGACAATGGAGTCCGGC |
| | ACATTCATCACCGACAAGTGCGTGCTGGATATGAAGGCTATGGACTCTAAGAGC |
| | AACGGCGCCATCGCCTGGAGCAATCAGACATCCTTCACCTGCCAGGATATCTTT |
| | AAGGAGACAAATGCCACCTACCCCAGCTCCGACGTGCCTTGTGATGCCACACTG |
| | ACCGAGAAGAGCTTCGAGACAGACATGAACCTGAATTTTCAGAACCTGCTGGTC |
| | ATCGTGCTGAGGATCCTGCTGCTGAAGGTGGCCGGCTTTAATCTGCTGATGACC |
| | CTGCGCCTGTGGTCTAGCTGA |
| 20 | ATGGCCTGCAGGCTGCTGTGCTGTGCCGTGCTGTGCCTGCTGGGAGCAGGAGAG |
| | CTGGTGCCAATGGAGACCGGAGTGACCCAGACACCAAGGCACCTGGTCATGGGC |
| | ATGACAAACAAGAAGAGCCTGAAGTGCGAGCAGCACCTGGGCCACAATGCCATG |
| | TACTGGTATAAGCAGTCCGCCAAGAAGCCTCTGGAGCTGATGTTCGTGTACTCT |
| | CTGGAGGAGCGGGTGGAGAACAATAGCGTGCCCAGCCGGTTCAGCCCAGAGTGC |
| | CCTAACAGCTCCCACCTGTTTCTGCACCTGCACACCCTGCAGCCAGAGGACTCC |
| | GCCCTGTACCTGTGCGCCTCTAGCCAGGACATCGGAGCAGGCAATACAGAGGCC |
| | TTCTTTGGCCAGGGCACCCGGCTGACAGTGGTGGAGGACCTGCGCAACGTGACA |
| | CCCCCTAAGGTGTCTCTGTTCGAGCCTAGCAAGGCCGAGATCGCCAATAAGCAG |
| | AAGGCCACCCTGGTGTGCCTGGCCAGAGGCTTCTTTCCAGATCACGTGGAGCTG |
| | TCCTGGTGGGTGAACGGCAAGGAGGTGCACTCTGGCGTGTGCACAGACCCCCAG |
| | GCCTACAAGGAGAGCAATTACTCCTATTGTCTGTCTAGCCGGCTGAGAGTGTCC |
| | GCCACCTTTTGGCACAACCCACGGAATCACTTCAGATGCCAGGTGCAGTTTCAC |
| | GGCCTGTCTGAGGAGGATAAGTGGCCAGAGGGAAGCCCAAAGCCAGTGACCCAG |
| | AACATCTCCGCCGAGGCATGGGGAAGGGCAGACTGTGGAATCACATCCGCCTCT |
| | TATCAGCAGGGCGTGCTGAGCGCCACCATCCTGTACGAGATCCTGCTGGGCAAG |
| | GCCACACTGTATGCCGTGCTGGTGTCCACCCTGGTGGTCATGGCTATGGTGAAG |
| | CGCAAGAACTCTAGGGCAAAGCGCAGCGGATCCGGAGCAACCAATTTCAGCCTG |
| | CTGAAGCAGGCAGGCGATGTGGAGGAGAACCCTGGACCAATGCTGCTGGAGCTG |
| | ATCCCTCTGCTGGGCATCCACTTCGTGCTGAGGACCGCCAGAGCCCAGTCCGTG |
| | ACACAGCCAGACATCCACATCACCGTGTCCGAGGGAGCCTCTCTGGAGCTGAGG |
| | TGCAACTACTCTTATGGCGCCACACCCTACCTGTTCTGGTACGTGCAGAGCCCT |
| | GGACAGGGACTGCAGCTGCTGCTGAAGTACTTTTCCGGCGACACCCTGGTGCAG |
| | GGCATCAAGGGCTTCGAGGCCGAGTTTAAGAGGTCTCAGAGCAGCTTCAACCTG |
| | CGCAAGCCAAGCGTGCACTGGTCCGATGCCGCCGAGTACTTCTGTGCCGTGTGG |
| | CCATATCAGGCAGGCACAGCCCTGATCTTTGGCAAGGGCACCACACTGAGCGTG |
| | TCTAGCAACATCCAGAATCCCGAGCCTGCCGTGTATCAGCTGAAGGACCCAAGA |
| | TCTCAGGATAGCACACTGTGCCTGTTCACCGACTTTGATAGCCAGATCAACGTG |
| | CCCAAGACAATGGAGTCCGGCACATTCATCACCGACAAGTGCGTGCTGGATATG |
| | AAGGCTATGGACTCTAAGAGCAACGGCGCCATCGCCTGGAGCAATCAGACATCC |
| | TTCACCTGCCAGGATATCTTTAAGGAGACAAATGCCACCTACCCCAGCTCCGAC |
| | GTGCCTTGTGATGCCACACTGACCGAGAAGAGCTTCGAGACAGACATGAACCTG |
| | AATTTTCAGAACCTGCTGGTCATCGTGCTGAGGATCCTGCTGCTGAAGGTGGCC |
| | GGCTTTAATCTGCTGATGACCCTGCGCCTGTGGTCTAGCTGA |
| 21 | ATGGCCATCAGGCTGCTGTGCCGCGTGGCCTTCTGTTTTCTGGCCGTGGGCCTG |
| | GTGGACGTGAAGGTGACCCAGAGCAGCAGATACCTGGTGAAGAGAACAGGCGAG |
| | AAGGTGTTCCTGGAGTGCGTGCAGGACATGGATCACGAGAACATGTTTTGGTAT |
| | CGGCAGGACCCCGGACTGGGACTGAGGCTGATCTACTTCTCTTATGACGTGAAG |
| | ATGAAGGAGAAGGGCGACATCCCTGAGGGCTACTCTGTGAGCAGGGAGAAGAAG |
| | GAGCGGTTCAGCCTGATCCTGGAGTCCGCCTCTACCAATCAGACAAGCATGTAC |
| | CTGTGCGCCTCCCGGAGACAGGGCACCGTGTACGAGCAGTATGTGGGACCCGGC |
| | ACAAGACTGACCGTGACAGAGGACCTGCGCAACGTGACACCCCCTAAGGTGTCT |
| | CTGTTCGAGCCTAGCAAGGCCGAGATCGCCAATAAGCAGAAGGCCACCCTGGTG |
| | TGCCTGGCCAGAGGCTTCTTTCCAGATCACGTGGAGCTGTCCTGGTGGGTGAAC |
| | GGCAAGGAGGTGCACTCTGGCGTGTGCACAGACCCCCAGGCCTACAAGGAGAGC |
| | AATTACTCCTATTGTCTGTCTAGCCGGCTGAGAGTGTCCGCCACCTTTTGGCAC |
| | AACCCACGGAATCACTTCAGATGCCAGGTGCAGTTTCACGGCCTGTCTGAGGAG |
| | GATAAGTGGCCAGAGGGAAGCCCAAAGCCAGTGACCCAGAACATCTCCGCCGAG |
| | GCATGGGGAAGGGCAGACTGTGGAATCACATCCGCCTCTTATCAGCAGGGCGTG |
| | CTGAGCGCCACCATCCTGTACGAGATCCTGCTGGGCAAGGCCACACTGTATGCC |
| | GTGCTGGTGTCCACCCTGGTGGTCATGGCTATGGTGAAGCGCAAGAACTCTAGG |
| | GCAAAGCGCAGCGGATCCGGAGCAACCAATTTCAGCCTGCTGAAGCAGGCAGGC |
| | GATGTGGAGGAGAACCCTGGACCAATGGAGAAGAACCCCCTGGCCGCACCTCTG |
| | CTGATCCTGTGGTTCCACCTGGACTGCGTGAGCAGCATCCTGAATGTGGAGCAG |
| | TCCCCACAGTCTCTGCACGTGCAGGAGGGCGATTCTACCAACTTCACATGTAGC |
| | TTTCCCTCTAGCAATTTCTATGCCCTGCACTGGTACAGGTGGGAGACCGCAAAG |
| | AGCCCTGAGGCCCTGTTTGTGATGACACTGAACGGCGACGAGAAGAAGAAGGGC |
| | CGGATCTCCGCCACCCTGAATACAAAGGAGGGCTACTCTTATCTGTACATCAAG |
| | GGCAGCCAGCCAGAGGATTCCGCCACCTATCTGTGCGCCTCCAACCGGGACGAT |
| | AAGATCATCTTTGGCAAGGGCACAAGACTGCACATCCTGCCCAACATCCAGAAT |
| | CCCGAGCCTGCCGTGTATCAGCTGAAGGACCCAAGATCTCAGGATAGCACACTG |
| | TGCCTGTTCACCGACTTTGATAGCCAGATCAACGTGCCCAAGACAATGGAGTCC |
| | GGCACATTCATCACCGACAAGTGCGTGCTGGATATGAAGGCTATGGACTCTAAG |
| | AGCAACGGCGCCATCGCCTGGAGCAATCAGACATCCTTCACCTGCCAGGATATC |
| | TTTAAGGAGACAAATGCCACCTACCCCAGCTCCGACGTGCCTTGTGATGCCACA |
| | CTGACCGAGAAGAGCTTCGAGACAGACATGAACCTGAATTTTCAGAACCTGCTG |
| | GTCATCGTGCTGAGGATCCTGCTGCTGAAGGTGGCCGGCTTTAATCTGCTGATG |
| | ACCCTGCGCCTGTGGTCTAGCTGA |
| 22 | NLVPMVATV |
| 23 | GAGAATCAAAATCGGTGAAT |
| 24 | GGGAATCAAAATCGGTGAAT |
| 25 | TSINN |
| 26 | IRSNERE |
| 27 | LGHDT |
| 28 | YNNKEL |
| 29 | YGATPY |
| 30 | YFSGDTLV |
| 31 | LGHNA |
| 32 | YSLEER |
| 33 | SSNFYA |
| 34 | MTLNGDE |
| 35 | MDHEN |
| 36 | SYDVKM |

*The CDR3 sequences are bolded in the variable region sequences, and the alpha and beta chain variable region sequences are bolded in the complete TCR sequences.

As detailed in Table 3, SEQ ID Nos 1-6 are CDR3 amino acid sequences of the alpha and beta chains of TCRs directed to hexon, pp65, and the minimal epitope SEQ ID NO. 22, respectively. SEQ ID Nos 7-12 are the respective variable chains amino acid sequences. SEQ ID Nos 13-18 are the respective variable chains DNA sequences. SEQ ID Nos. 19-21 are the complete constructs used in the examples. SEQ ID No 22: the minimal CMV pp65 epitope NLVPMVATV. SEQ ID NO: 23, 24 are gRNAs used with CRISPR. SEQ ID Nos 25-36 are sequences of CDR1 and 2 of the alpha and beta chains of TCRs directed to hexon, pp65, and the minimal epitope SEQ ID NO. 22, respectively. All CDR sequences are provided as amino acid sequences, and their corresponding nucleic acid sequence can easily be found from the corresponding Vnuc sequences.

TABLE 3
details of TCR sequences of three rTCR-Vs

| | | | | | |
|---|---|---|---|---|---|
| TCR | | | | Region | SEQ |
| chain | V | D | J | SEQ | ID |

Construct 1: ADV, hexon protein

| | | | | | |
|---|---|---|---|---|---|
| alpha | TRAV17*01 | — | TRAJ57*01 | CDR1 | 25 |
| | | | | CDR2 | 26 |
| | | | | CDR3 | 1 |
| | | | | Vaa | 7 |
| | | | | Vnuc | 13 |
| beta | TRBV3-1*01 | TRBD2*01 | TRBJ1-1*01 | CDR1 | 27 |
| | | | | CDR2 | 28 |
| | | | | CDR3 | 2 |
| | | | | Vaa | 8 |
| | | | | Vnuc | 14 |
| all | | | | TCRnuc | 19 |

Construct 2: CMV, pp65 protein in DPB1*: 04:01

| | | | | | |
|---|---|---|---|---|---|
| alpha | TRAV8-3*01 | — | TRAJ15*01 | CDR1 | 29 |
| | | | | CDR2 | 30 |
| | | | | CDR3 | 3 |
| | | | | Vaa | 9 |
| | | | | Vnuc | 15 |
| beta | TRBV4-3*01 | TRBD2*02 | TRBJ1-1*01 | CDR1 | 31 |
| | | | | CDR2 | 32 |
| | | | | CDR3 | 4 |
| | | | | Vaa | 10 |
| | | | | Vnuc | 16 |
| all | | | | TCRnuc | 20 |

Construct 3: CMV, pp65 protein minimal epitope
(SEQ ID NO: 22) in HLA A 02:01

| | | | | | |
|---|---|---|---|---|---|
| alpha | TRAV24*01 | — | TRAJ30*01 | CDR1 | 33 |
| | | | | CDR2 | 34 |
| | | | | CDR3 | 5 |
| | | | | Vaa | 11 |
| | | | | Vnuc | 17 |
| beta | TRBV28*01 | TRBD1*01 | TRBJ2-7*02 | CDR1 | 35 |
| | | | | CDR2 | 36 |
| | | | | CDR3 | 6 |
| | | | | Vaa | 12 |
| | | | | Vnuc | 18 |
| all | | | | TCRnuc | 21 |

CDR1/2/3: amino acid sequence of the CDR3; Vaa: amino acid sequence of the variable region; Vnuc: nucleic acid sequence of the variable region; TCR: full length TCR nucleotide sequence, prepared as described below.

Generally, the antigen-specific TCR variable region sequences were reconstructed to full-length TCR Vβ/Vα chains, cloned into the retroviral vector MSGV1, transduced into autologous or allogeneic T-cells, and tested against antigen-presenting cells loaded with the specific epitopes.

Specifically, DNA sequences encoding the T cell receptor alpha (TRA) V-J region (SEQ ID Nos: 13, 15, 17, see Tables 2, 3) were fused to a mouse TCR-α constant chain, and DNA sequences encoding the T cell receptor beta (TRB) V-D-J region (SEQ ID Nos: 14, 16, 18, see Tables 2, 3) were fused to mouse TCR-β constant chain, as described in Cohen et al., 2006, Enhanced antitumor activity of murine-human hybrid T-cell receptor (TCR) in human lymphocytes is associated with improved pairing and TCR/CD3 stability. Cancer Res 66: 8878-8886). The full-length TRB and TRA chains were joined together with a furin SGSG P2A linker separating them (beta chain—furin linker—alpha chain, SEQ ID Nos: 19-21). The complete TCR construct was cloned into a pMSGV 1 retroviral vector. For transduction, autologous or allogeneic white blood cells from leukapheresis samples were thawed and set to 2×10⁶ cells/ml in T-cell medium, which consists of a 50/50 mixture of RPMI and AIM-V media supplemented with 5% in-house human serum, 10 μg/ml gentamicin (CellGro), 100 U/ml penicillin and 100 μg/ml streptomycin, and 2 mM L-glutamine (all from Life Technologies). 2×10⁶ cells/ml were stimulated in a 24-well plate with 50 ng/ml soluble anti-CD3 antibody OKT3 (Miltenyi Biotec) and 300 IU/ml IL-2 (Chiron) for 2 days before retroviral transduction. Retroviral supernatants were generated in HEK-293GP packaging line. Briefly, pMSGV1 plasmid encoding the specific TCR (2 μg/well) and the envelope-encoding plasmid RD114 (0.75 μg/well) were co-transfected into 1×10⁶ 239GP cells/well of a 6-well poly-D-lysine-coated plates using Lipofectamine 2000 (Life Technologies). Retroviral supernatants were collected at 42-48 hours after transfection, diluted 1:1 with DMEM media, and centrifuged onto Retronectin-coated (10 μg/ml, Takara), non-tissue culture-treated 6-well plates at 2,000 g for 2 hours at 32° C. Stimulated T-cells (2×10⁶ cells/well, at 0.5×10⁶ cells/ml in IL-2 containing T-cell media) were then spun onto the retrovirus plates for 10 minutes at 300-350 G. Stimulated T-cells were transduced overnight, removed from the plates, and further cultured in rIL-2 containing T-cell media. GFP and mock transduction controls were included in transduction experiments. Cells were typically assayed 10-14 days post-retroviral transduction. FIGS. 5J-5L show the activity of CMV-specific TCRs against the antigen-presenting cells loaded with pp65 peptides, conducted as described above. As can be shown, T cells comprising a TCR specific to the antigen, for example CMV-pp65 (FIG. 5K) and minimal CMV-pp65 epitope of SEQ ID NO: 22 (FIG. 5L) were activated, as can be seen by the increased expression of 4-1BB and/or OX40 compared to the no TCR controls (FIG. 5J). As can also be seen, both CD4⁺ (left panels) and CD8⁺ (right panels) cells were activated compared to the no TCR controls.

Thus, the results demonstrate the ability to produce T cells comprising a recombinant TCR specific for viral peptides, herein named TCR-V T-cells.

### Example 5: Infection of Primary Donor Monocytes with Live Adenovirus (ADV)

A method to infect primary donor monocytes with live viruses was developed, in order to facilitate to functionally test the ability of the TCR-V T-cells to recognize and kill autologous donor tissue infected with a relevant virus. For functional validation, the method includes isolating monocytes from donor PBMCs harboring relevant HLA molecules, infecting the monocytes with a live virus, and after 24 hours co-incubating the virus-infected monocytes with TCR-V T-cells directed against the same virus. Specific T-cell activation may then be measured by secretion of IFNγ following virus recognition.

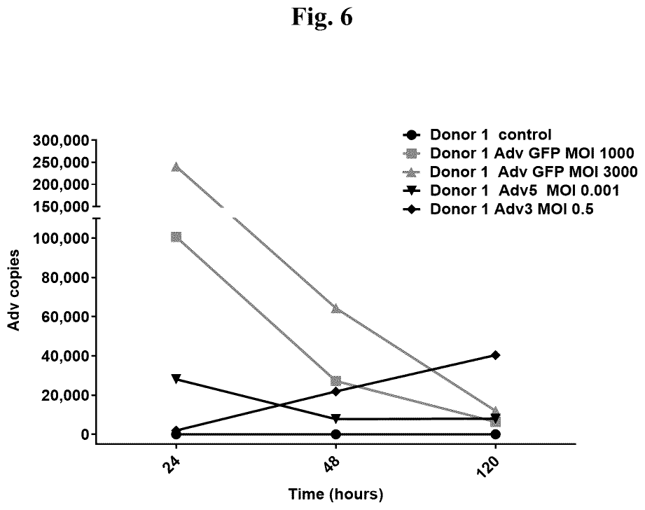

The method was tested with ADV. To establish the ADV infection procedure, three viral samples were used: an ADV-GFP system and two additional ADV subtypes (ADV5 and ADV3) isolated by the national virology laboratory from patients suffering from ADV infections. The results presented in FIG. 6 demonstrate that PBMCs can be efficiently infected with different ADV variants. The differences in ADV copies between the ADV-GFP and the ADV5/3 variants result from a lower concentration of the virus.

### Example 6: in vitro Stimulation (IVS) in a Large Scale

TABLE 3
Reagents

| | | manufacturer/ |
|---|---|---|
| Reagent | Catalog | Distributer |
| Ficoll ® Lymphoprep | 04-03-9391 | Alere tech/ |
| AIM V ™ medium (GMP) | 87-0112BK | Gibco/Rhenium |
| Human serum | #HP1022 | Valley Biomedical, |
| | | Winchester, VA, USA |
| GlutaMAX 200 mM (L- | A1286001 | Gibco/Rhenium |
| glutamine) | | |
| Pen/Strep (penicillin, 10⁴ | 15140122 | Gibco/Rehnium |
| U/ml, streptomycin, 10⁴ | | |
| μg/ml) | | |
| Recombinant human IL2, | 5060229220264 | Clinigen/Sheba |
| Proleukin ® (aldesleukin) | | pharmacy |
| (drug) | | |
| Hexon (Adv) Peptide library | 170-076-106 | Miltenyi/Almog |
| (clinical grade) | | |
| LTA (BKV) Peptide library | 170-076-139 | Miltenyi/Almog |
| (clinical grade) | | |
| VP1 (BKV) Peptide library | 170-076-138 | Miltenyi/Almog |
| (clinical grade) | | |
| pp65 (CMV) Peptide library | 170-076-109 | Miltenyi/Almog |
| (clinical grade) | | |
| DPBS | 02-023-1A | Biological industries |
| NutriFreeZ D10 | 05-713-1A | Biological industries |
| CryoSure-DEX40 | 0482 | WAK-Chemie medical |
| | | GmbH/Almog |
| Zenalb- Human serum | 901064 | BPL/Sheba pharmacy |
| albumin (HSA) | | |

PBMCs were isolated as described above, and suspended in VST medium (2 mM GlutaMAX, 5% Heat inactivated human serum in AIM-V™ medium). 150×10⁶ cells in 1,840 μl VST medium were plated into one well in a 6-well plate (ultra-low attachment surface).

Single peptide libraries for hexon (ADV), VP1 (BKV), LTA (BKV) and pp65 (CMV) were used to prepare a pooled peptide library master mix by mixing equal volumes from each 50 g/ml stock. 160 μl of the pooled peptide library master mix were added to the well (6 well) containing the PBMCs, and incubate the cells at 37° C., 500 CO₂ for 2 hours.

After 2 hours of priming, cells were transferred to a G-REX® 100M production platform (Wilson Wolf, Cat. #P/N 81100) containing warm VST medium (37° C.), and incubated for 3 days at 37° C., 5% CO2. VST medium containing IL2 at a final concentration of 300 IU/ml was then added to the G-REX® and the culture continued to grow for 8-9 days at 37° C., 5% CO₂.

Cells from the G-REX® membrane were resuspended and collected. After centrifugation, the cells were resuspended in PBS containing 5.7% human serum albumin. Cells were then counted with a hemi-cytometer and Trypan blue and analyzed by FACS. 20-50×10⁶ remaining cells may be transferred to freezing tubes in freezing medium for cryopreservation.

### Example 7: Generation of TCR/CD3 Negative T Cells by CRISPR-Cas9 System

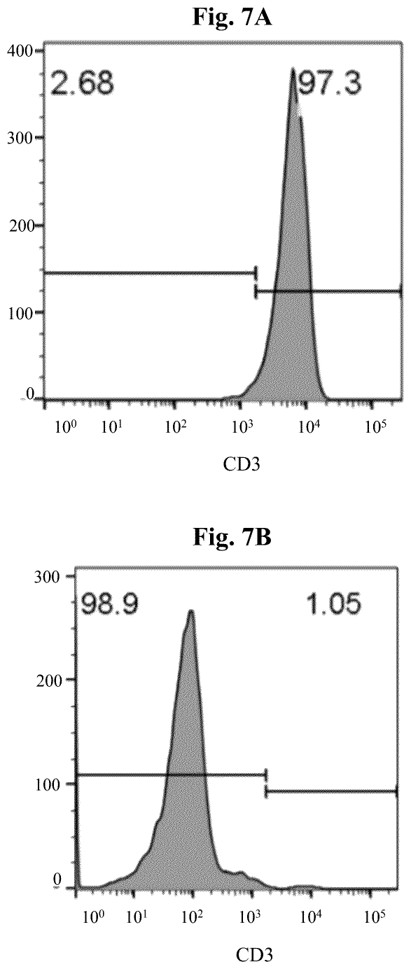

In order to avoid alloreactivity and a possible graft versus host disease (GvHD), the inventors developed CRISPR-Cas9 genome editing to knock-out (KO) endogenous TCRα in TCR-V T-cells. A published gRNA, SEQ ID NO: 23, GAGAAUCAAAAUCGGUGAAU (Morton et al. 2020, Simultaneous Deletion of Endogenous TCRalphabeta for TCR Gene Therapy Creates an Improved and Safe Cellular Therapeutic. Mol Ther. 28(1):64-74) was used to target the T Cell Receptor Alpha Constant (TRAC) gene in T-cells derived from peripheral-blood mononuclear cells (PBMCs), As described therein. Briefly, T-cells were isolated from PBMCs as described above, stimulated for 2 days by using CD3/CD28 beads (Dynabeads™ Human T-Activator CD3/CD28 for T Cell Expansion and Activation), according to the manufacturer's instructions, and cultured in an IL2-containing medium, as described above, to facilitate proliferation. On day 2 of the culture, the cells were harvested and subjected to electroporation with an RNP complex containing CRISPR-cas9 and the SEQ ID NO: 23 gRNA for targeting the TRAC locus. Subsequently, the cells were cultured for an additional 5 days using the same medium, after which they were collected for a FACS analysis, and stained with an anti-CD3 antibody (Miltenyi Biotec). As can be seen from FIGS. 7A-7B, in contrast to untreated cells, which exhibit a high expression of CD3 (97%, FIG. 7A), the manipulated cells demonstrated a significant reduction in TCR expression, with CD3 levels decreasing to approximately 1% (FIG. 7B).

### Example 8: Off-Target Analysis of TRAC gRNAs Using GUIDE-Seq and rhAmpSeq

For therapeutic applications, unwanted genome-editing outcomes need to be defined by the most sensitive, unbiased methods, given that even low-frequency events in large populations of cells could have devastating consequences.

For that purpose, the inventors have developed a methodology for the unbiased identification and quantification of bona-fide off-target sites based on the combination of GUIDE-Seq (Tsai et al. 2015, GUIDE-seq enables genome-wide profiling of off-target cleavage by CRISPR-Cas nucleases, Nat Biotechnol. 33(2):187-97) in cells with stable expression of Cas9, and the orthogonal rhAmpSeq technology (Dobosy et al. 2011, RNase H-dependent PCR (rhPCR): improved specificity and single nucleotide polymorphism detection using blocked cleavable primers. BMC Biotechnol. 11:80) in primary cells. This methodology was tested for identifying off-target sites in two TRAC-targeting gRNAs from available publications, herein designated SEQ ID NO: 23 (GAGAATCAAAATCGGTGAAT) and SEQ ID NO: 24 (GGGAATCAAAATCGGTGAAT) (Dobosy et al. 2011 and Kath et al. 2022, Pharmacological interventions enhance virus-free generation of TRAC-replaced CAR T cells. Mol Ther Methods Clin Dev. 25:311-30). By off-target sequence alignment visualization using GUIDE-seq data, two and six putative off-target sites were found for SEQ ID NO: 23 and SEQ ID NO: 24, respectively, following the delivery of the two TRAC-targeting gRNAs into HEK293 cells with stable expression of Cas9 (data not shown).

### Example 9: Mixed Lymphocyte Reaction to Track Alloresponse

To evaluate the lack of allogeneic response of the knocked-out cells, a mixed lymphocyte reaction (MLR) assay is conducted. The reaction is set between TCR-V T-cells that were edited by CRISPR to knock-out endogenous TCR, as described below, and stained with the fluorescent dye (carboxyfluorescein succinimidyl ester (CFSE) (responder cells), and PBMCs derived from allogeneic donors (stimulator cells). Proliferation is measured to evaluate activation following the allogeneic recognition.

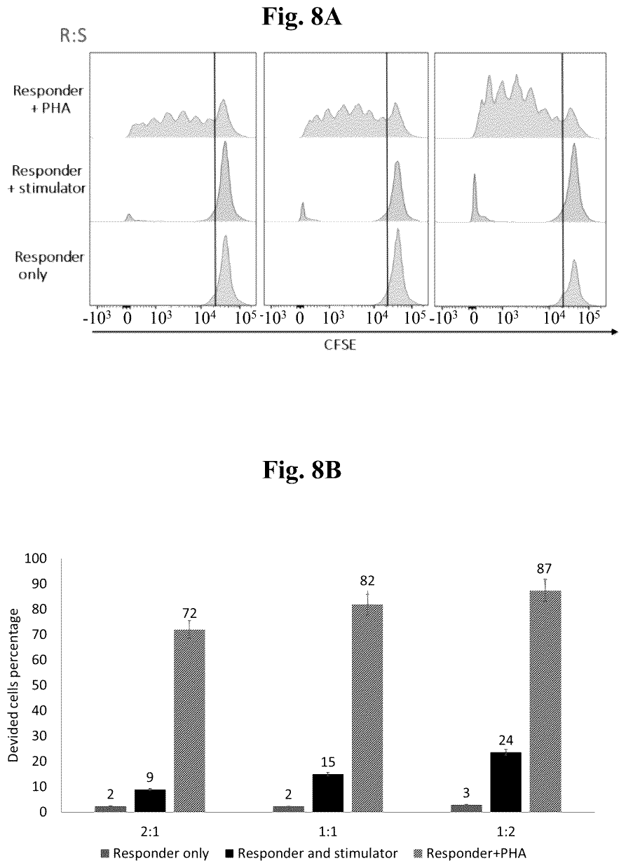

As a proof of concept, PBMCs stained with CSFE (responder cells) were incubated with irradiated PBMCs (stimulator cells). As shown in FIGS. 8A-8B, there is a strong allogeneic response to allogeneic donor cells, as seen from the loss of CFSE staining, which represents T cells in a proliferation stage. The strength of the response appears to inversely correlated with the responder-stimulator (R:S) ratio.

1-51. (canceled)

52. A recombinant virus-specific T cell receptor (rTCR-V) specific to a cytomegalovirus (CMV) or an adenovirus (ADV) viral antigen restricted by a predetermined HLA type, the rTCR-V comprising six CDRs having sequences selected from the group consisting of:
a. SEQ ID NOs 33, 34, 5, 35, 36, and 6;
b. SEQ ID NOs 29, 30, 3, 31, 32, and 4; and
c. SEQ ID NOs 25, 26, 1, 27, 28, and 2.

53. The recombinant virus-specific T cell receptor (rTCR-V) of claim 52, comprising alpha+beta variable region sequences selected from the group consisting of: SEQ ID NOs: 11+12; SEQ ID NOs: 9+10; and SEQ ID NOs: 7+8.

54. A composition comprising T cells expressing the recombinant virus-specific T cell receptor (rTCR-V) of claim 52.

55. The composition of claim 54, wherein the T cells comprise CD4⁺ T cells.

56. The composition of claim 54, wherein the T cells comprise CD8⁺ T cells.

57. The composition of claim 54, wherein the T cells do not express an endogenous TCR.

58. The composition of claim 54, wherein the T cells have an HLA type different from the predetermined HLA type.

59. The composition of claim 54, wherein the rTCR-V comprises only sequences exogenous to the T cells.

60. The composition of claim 54, wherein the rTCR-V comprises a TCR sequence endogenous to the T cell in which a variable region sequence is replaced by a corresponding virus-specific TCR sequence exogenous to the T cell, the exogenous sequence comprising at least a virus-specific alpha chain CDR3 region sequence and/or a virus-specific beta chain CDR3 region sequence.

61. The composition of claim 59, wherein the T cells comprise a knocked-out (KO) TCR alpha constant (TRAC) locus and a sequence encoding the exogenous sequence is inserted into the TRAC locus on the T cells.

62. The composition of claim 54, further comprising a pharmaceutically acceptable carrier.

63. A method of treating or preventing viral infection in a subject, the method comprising administering to the subject a therapeutically effective amount of the composition of claim 62.

64. The method of claim 63, wherein the treating or preventing is conducted by adoptive cell therapy (ACT).

65. The method of claim 63, wherein the administration is following transplantation of an organ or cells from a transplantation donor, such as a hematopoietic stem cell transplantation (HSCT) or a solid organ transplantation (SOT) from a donor.

66. The method of claim 63, wherein the T cells are not derived from the subject or the transplantation donor.

67. The method of claim 63, wherein the subject is infected with a virus selected from the group consisting of adenovirus (ADV) and cytomegalovirus (CMV).


SEQUENCE LISTING (36 sequences)

SEQ ID NO: 1 - protein, 14 aa, Homo sapiens
CATDRGGGSEKLVF

SEQ ID NO: 2 - protein, 13 aa, Homo sapiens
CASSQLAGHEAFF

SEQ ID NO: 3 - protein, 14 aa, Homo sapiens
CAVWPYQAGTALIF

SEQ ID NO: 4 - protein, 16 aa, Homo sapiens
CASSQDIGAGNTEAFF

SEQ ID NO: 5 - protein, 11 aa, Homo sapiens
CASNRDDKIIF

SEQ ID NO: 6 - protein, 14 aa, Homo sapiens
CASRRQGTVYEQYV

SEQ ID NO: 7 - protein, 132 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDRGGGSEKLVFGKGTKLTVNP

SEQ ID NO: 8 - protein, 131 aa, Homo sapiens
MACRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQLAGHEAFFGQGTRLTVV

SEQ ID NO: 9 - protein, 133 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVWPYQAGTALIFGKGTTLSVSS

SEQ ID NO: 10 - protein, 137 aa, Homo sapiens
MACRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDIGAGNTEAFFGQGTRLTVV

SEQ ID NO: 11 - protein, 132 aa, Homo sapiens
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCASNRDDKIIFGKGTRLHILP

SEQ ID NO: 12 - protein, 132 aa, Homo sapiens
MAIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRRQGTVYEQYVGPGTRLTVT

SEQ ID NO: 13 - DNA, 396 bp, Homo sapiens
  source 1..396: mol_type = other DNA
atggagacactgctgggcgtgagcctggtcatcctgtggctgcagctggcccgggtgaacagccagcagggagaggaggacccccaggccctgtccatccaggagggcgagaacgccaccatgaattgcagctacaagacatccatcaacaatctgcagtggtatcggcagaactctggcagaggcctggtgcacctgatcctgatcaggtctaatgagcgcgagaagcacagcggccggctgagagtgaccctggatacaagcaagaagagcagcagcctgctgatcaccgcctccagggccgcagacacagcctcttacttctgtgcaaccgataggggaggaggctccgagaagctggtgtttggcaagggcaccaagctgacagtgaatcct

SEQ ID NO: 14 - DNA, 393 bp, Homo sapiens
  source 1..393: mol_type = other DNA
atggcctgccggctgctgtgctgcgtggtgttctgcctgctgcaggccggcccactggacaccgccgtgtctcagacacccaagtacctggtgacccagatgggcaacgataagtccatcaagtgcgagcagaatctgggccacgacacaatgtactggtataagcaggatagcaagaagttcctgaagatcatgttttcctataacaataaggagctgatcatcaacgagaccgtgcccaatcggttctctcccaagagccctgacaaggcccacctgaacctgcacatcaattccctggagctgggcgattctgccgtgtacttttgtgccagctcccagctggccggacacgaggccttctttggacagggcaccagactgacagtggtg

SEQ ID NO: 15 - DNA, 399 bp, Homo sapiens
  source 1..399: mol_type = other DNA
atgctgctggagctgatccctctgctgggcatccacttcgtgctgaggaccgccagagcccagtccgtgacacagccagacatccacatcaccgtgtccgagggagcctctctggagctgaggtgcaactactcttatggcgccacaccctacctgttctggtacgtgcagagccctggacagggactgcagctgctgctgaagtacttttccggcgacaccctggtgcagggcatcaagggcttcgaggccgagtttaagaggtctcagagcagcttcaacctgcgcaagccaagcgtgcactggtccgatgccgccgagtacttctgtgccgtgtggccatatcaggcaggcacagccctgatctttggcaagggcaccacactgagcgtgtctagc

SEQ ID NO: 16 - DNA, 410 bp, Homo sapiens
  source 1..410: mol_type = other DNA
atggcctgcaggctgctgtgctgtgccgtgctgtgcctgctgggagcaggagagctggtgccaatggagaccggagtgacccagacaccaaggcacctggtcatgggcatgacaaacaagaagagcctgaagtgcgagcagcacctgggccacaatgccatgtactggtataagcagtccgccaagaagcctctggagctgatgttcgtgtactctctggaggagcgggtggagaacaatagcgtgcccagccggttcagcccagagtgccctaacagctcccacctgtttctgcacctgcacaccctgcagccagaggactccgccctgtacctgtgcgcctctagccaggacatcggagcaggcaatacagaggccttctttggccagggcacccggctgacagtggt

SEQ ID NO: 17 - DNA, 396 bp, Homo sapiens
  source 1..396: mol_type = other DNA
atggagaagaaccccctggccgcacctctgctgatcctgtggttccacctggactgcgtgagcagcatcctgaatgtggagcagtccccacagtctctgcacgtgcaggagggcgattctaccaacttcacatgtagctttccctctagcaatttctatgccctgcactggtacaggtgggagaccgcaaagagccctgaggccctgtttgtgatgacactgaacggcgacgagaagaagaagggccggatctccgccaccctgaatacaaaggagggctactcttatctgtacatcaagggcagccagccagaggattccgccacctatctgtgcgcctccaaccgggacgataagatcatctttggcaagggcacaagactgcacatcctgccc

SEQ ID NO: 18 - DNA, 396 bp, Homo sapiens
  source 1..396: mol_type = other DNA
atggccatcaggctgctgtgccgcgtggccttctgttttctggccgtgggcctggtggacgtgaaggtgacccagagcagcagatacctggtgaagagaacaggcgagaaggtgttcctggagtgcgtgcaggacatggatcacgagaacatgttttggtatcggcaggaccccggactgggactgaggctgatctacttctcttatgacgtgaagatgaaggagaagggcgacatccctgagggctactctgtgagcagggagaagaaggagcggttcagcctgatcctggagtccgcctctaccaatcagacaagcatgtacctgtgcgcctcccggagacagggcaccgtgtacgagcagtatgtgggacccggcacaagactgaccgtgaca

SEQ ID NO: 19 - DNA, 1803 bp, synthetic construct
  source 1..1803: mol_type = other DNA
atggcctgccggctgctgtgctgcgtggtgttctgcctgctgcaggccggcccactggacaccgccgtgtctcagacacccaagtacctggtgacccagatgggcaacgataagtccatcaagtgcgagcagaatctgggccacgacacaatgtactggtataagcaggatagcaagaagttcctgaagatcatgttttcctataacaataaggagctgatcatcaacgagaccgtgcccaatcggttctctcccaagagccctgacaaggcccacctgaacctgcacatcaattccctggagctgggcgattctgccgtgtacttttgtgccagctcccagctggccggacacgaggccttctttggacagggcaccagactgacagtggtggaggacctgcgcaacgtgacaccccctaaggtgtctctgttcgagcctagcaaggccgagatcgccaataagcagaaggccaccctggtgtgcctggccagaggcttctttccagatcacgtggagctgtcctggtgggtgaacggcaaggaggtgcactctggcgtgtgcacagacccccaggcctacaaggagagcaattactcctattgtctgtctagccggctgagagtgtccgccaccttttggcacaacccacggaatcacttcagatgccaggtgcagtttcacggcctgtctgaggaggataagtggccagagggaagcccaaagccagtgacccagaacatctccgccgaggcatggggaagggcagactgtggaatcacatccgcctcttatcagcagggcgtgctgagcgccaccatcctgtacgagatcctgctgggcaaggccacactgtatgccgtgctggtgtccaccctggtggtcatggctatggtgaagcgcaagaactctagggcaaagcgcagcggatccggagcaaccaatttcagcctgctgaagcaggcaggcgatgtggaggagaaccctggaccaatggagacactgctgggcgtgagcctggtcatcctgtggctgcagctggcccgggtgaacagccagcagggagaggaggacccccaggccctgtccatccaggagggcgagaacgccaccatgaattgcagctacaagacatccatcaacaatctgcagtggtatcggcagaactctggcagaggcctggtgcacctgatcctgatcaggtctaatgagcgcgagaagcacagcggccggctgagagtgaccctggatacaagcaagaagagcagcagcctgctgatcaccgcctccagggccgcagacacagcctcttacttctgtgcaaccgataggggaggaggctccgagaagctggtgtttggcaagggcaccaagctgacagtgaatcctaacatccagaatcccgagcctgccgtgtatcagctgaaggacccaagatctcaggatagcacactgtgcctgttcaccgactttgatagccagatcaacgtgcccaagacaatggagtccggcacattcatcaccgacaagtgcgtgctggatatgaaggctatggactctaagagcaacggcgccatcgcctggagcaatcagacatccttcacctgccaggatatctttaaggagacaaatgccacctaccccagctccgacgtgccttgtgatgccacactgaccgagaagagcttcgagacagacatgaacctgaattttcagaacctgctggtcatcgtgctgaggatcctgctgctgaaggtggccggctttaatctgctgatgaccctgcgcctgtggtctagctga

SEQ ID NO: 20 - DNA, 1824 bp, synthetic construct
  source 1..1824: mol_type = other DNA
atggcctgcaggctgctgtgctgtgccgtgctgtgcctgctgggagcaggagagctggtgccaatggagaccggagtgacccagacaccaaggcacctggtcatgggcatgacaaacaagaagagcctgaagtgcgagcagcacctgggccacaatgccatgtactggtataagcagtccgccaagaagcctctggagctgatgttcgtgtactctctggaggagcgggtggagaacaatagcgtgcccagccggttcagcccagagtgccctaacagctcccacctgtttctgcacctgcacaccctgcagccagaggactccgccctgtacctgtgcgcctctagccaggacatcggagcaggcaatacagaggccttctttggccagggcacccggctgacagtggtggaggacctgcgcaacgtgacaccccctaaggtgtctctgttcgagcctagcaaggccgagatcgccaataagcagaaggccaccctggtgtgcctggccagaggcttctttccagatcacgtggagctgtcctggtgggtgaacggcaaggaggtgcactctggcgtgtgcacagacccccaggcctacaaggagagcaattactcctattgtctgtctagccggctgagagtgtccgccaccttttggcacaacccacggaatcacttcagatgccaggtgcagtttcacggcctgtctgaggaggataagtggccagagggaagcccaaagccagtgacccagaacatctccgccgaggcatggggaagggcagactgtggaatcacatccgcctcttatcagcagggcgtgctgagcgccaccatcctgtacgagatcctgctgggcaaggccacactgtatgccgtgctggtgtccaccctggtggtcatggctatggtgaagcgcaagaactctagggcaaagcgcagcggatccggagcaaccaatttcagcctgctgaagcaggcaggcgatgtggaggagaaccctggaccaatgctgctggagctgatccctctgctgggcatccacttcgtgctgaggaccgccagagcccagtccgtgacacagccagacatccacatcaccgtgtccgagggagcctctctggagctgaggtgcaactactcttatggcgccacaccctacctgttctggtacgtgcagagccctggacagggactgcagctgctgctgaagtacttttccggcgacaccctggtgcagggcatcaagggcttcgaggccgagtttaagaggtctcagagcagcttcaacctgcgcaagccaagcgtgcactggtccgatgccgccgagtacttctgtgccgtgtggccatatcaggcaggcacagccctgatctttggcaagggcaccacactgagcgtgtctagcaacatccagaatcccgagcctgccgtgtatcagctgaaggacccaagatctcaggatagcacactgtgcctgttcaccgactttgatagccagatcaacgtgcccaagacaatggagtccggcacattcatcaccgacaagtgcgtgctggatatgaaggctatggactctaagagcaacggcgccatcgcctggagcaatcagacatccttcacctgccaggatatctttaaggagacaaatgccacctaccccagctccgacgtgccttgtgatgccacactgaccgagaagagcttcgagacagacatgaacctgaattttcagaacctgctggtcatcgtgctgaggatcctgctgctgaaggtggccggctttaatctgctgatgaccctgcgcctgtggtctagctga

SEQ ID NO: 21 - DNA, 1806 bp, synthetic construct
  source 1..1806: mol_type = other DNA
atggccatcaggctgctgtgccgcgtggccttctgttttctggccgtgggcctggtggacgtgaaggtgacccagagcagcagatacctggtgaagagaacaggcgagaaggtgttcctggagtgcgtgcaggacatggatcacgagaacatgttttggtatcggcaggaccccggactgggactgaggctgatctacttctcttatgacgtgaagatgaaggagaagggcgacatccctgagggctactctgtgagcagggagaagaaggagcggttcagcctgatcctggagtccgcctctaccaatcagacaagcatgtacctgtgcgcctcccggagacagggcaccgtgtacgagcagtatgtgggacccggcacaagactgaccgtgacagaggacctgcgcaacgtgacaccccctaaggtgtctctgttcgagcctagcaaggccgagatcgccaataagcagaaggccaccctggtgtgcctggccagaggcttctttccagatcacgtggagctgtcctggtgggtgaacggcaaggaggtgcactctggcgtgtgcacagacccccaggcctacaaggagagcaattactcctattgtctgtctagccggctgagagtgtccgccaccttttggcacaacccacggaatcacttcagatgccaggtgcagtttcacggcctgtctgaggaggataagtggccagagggaagcccaaagccagtgacccagaacatctccgccgaggcatggggaagggcagactgtggaatcacatccgcctcttatcagcagggcgtgctgagcgccaccatcctgtacgagatcctgctgggcaaggccacactgtatgccgtgctggtgtccaccctggtggtcatggctatggtgaagcgcaagaactctagggcaaagcgcagcggatccggagcaaccaatttcagcctgctgaagcaggcaggcgatgtggaggagaaccctggaccaatggagaagaaccccctggccgcacctctgctgatcctgtggttccacctggactgcgtgagcagcatcctgaatgtggagcagtccccacagtctctgcacgtgcaggagggcgattctaccaacttcacatgtagctttccctctagcaatttctatgccctgcactggtacaggtgggagaccgcaaagagccctgaggccctgtttgtgatgacactgaacggcgacgagaagaagaagggccggatctccgccaccctgaatacaaaggagggctactcttatctgtacatcaagggcagccagccagaggattccgccacctatctgtgcgcctccaaccgggacgataagatcatctttggcaagggcacaagactgcacatcctgcccaacatccagaatcccgagcctgccgtgtatcagctgaaggacccaagatctcaggatagcacactgtgcctgttcaccgactttgatagccagatcaacgtgcccaagacaatggagtccggcacattcatcaccgacaagtgcgtgctggatatgaaggctatggactctaagagcaacggcgccatcgcctggagcaatcagacatccttcacctgccaggatatctttaaggagacaaatgccacctaccccagctccgacgtgccttgtgatgccacactgaccgagaagagcttcgagacagacatgaacctgaattttcagaacctgctggtcatcgtgctgaggatcctgctgctgaaggtggccggctttaatctgctgatgaccctgcgcctgtggtctagctga

SEQ ID NO: 22 - protein, 9 aa, synthetic construct
NLVPMVATV

SEQ ID NO: 23 - RNA, 20 nt, synthetic construct
  source 1..20: mol_type = other RNA
gagaatcaaaatcggtgaat

SEQ ID NO: 24 - RNA, 20 nt, synthetic construct
  source 1..20: mol_type = other RNA
gggaatcaaaatcggtgaat

SEQ ID NO: 25 - protein, 5 aa, Homo sapiens
TSINN

SEQ ID NO: 26 - protein, 7 aa, Homo sapiens
IRSNERE

SEQ ID NO: 27 - protein, 5 aa, Homo sapiens
LGHDT

SEQ ID NO: 28 - protein, 6 aa, Homo sapiens
YNNKEL

SEQ ID NO: 29 - protein, 6 aa, Homo sapiens
YGATPY

SEQ ID NO: 30 - protein, 8 aa, Homo sapiens
YFSGDTLV

SEQ ID NO: 31 - protein, 5 aa, Homo sapiens
LGHNA

SEQ ID NO: 32 - protein, 6 aa, Homo sapiens
YSLEER

SEQ ID NO: 33 - protein, 6 aa, Homo sapiens
SSNFYA

SEQ ID NO: 34 - protein, 7 aa, Homo sapiens
MTLNGDE

SEQ ID NO: 35 - protein, 5 aa, Homo sapiens
MDHEN

SEQ ID NO: 36 - protein, 6 aa, Homo sapiens
SYDVKM